(19) United States
(12) Patent Application Publication
(10) Pub. No.: US 2024/0378459 A1
(43) Pub. Date: Nov. 14, 2024

(54) DNA-BASED DIGITAL INFORMATION STORAGE WITH SIDEWALL ELECTRODES

(71) Applicant: Twist Bioscience Corporation, South San Francisco, CA (US)

(72) Inventors: Brian Wayne BRAMLETT, Portland, OR (US); Bill James PECK, Santa Clara, CA (US)

(73) Assignee: Twist Bioscience Corporation, South San Francisco, CA (US)

(21) Appl. No.: 18/781,511

(22) Filed: Jul. 23, 2024

Related U.S. Application Data
(63) Continuation of application No. 17/154,879, filed on Jan. 21, 2021, now Pat. No. 12,086,722.
Application No. 17/154,879 is a continuation of application No. 16/239,453, filed on Jan. 3, 2019, now Pat. No. 10,936,953.
(60) Provisional application No. 62/613,728, filed on Jan. 4, 2018.
(60) Provisional application No. 62/617,067, filed on Jan. 12, 2018.
(60) Provisional application No. 62/650,321, filed on Mar. 30, 2018.

(51) Int. Cl. G06N 3/123; B01J 19/00; C12N 15/09; C12N 15/10; C12Q 1/6869; C40B 50/14; G06F 16/00; G06F 17/40; G06N 99/00; G11C 11/00; G11C 11/56; G11C 13/00; G11C 13/02; G16B 30/00; G16B 50/20

(52) U.S. Cl. CPC G06N 3/123; B01J 19/0046; C12N 15/09; C12N 15/1068; C40B 50/14; G06F 17/40; G06N 99/007; G11C 11/00; G11C 11/56; G11C 13/0019; G11C 13/02; G16B 50/20; B01J 2219/00313; B01J 2219/0059; B01J 2219/00653; B01J 2219/00722; C12Q 1/6869; G06F 16/00; G16B 30/00

(57) ABSTRACT

Provided herein are compositions, devices, systems and methods for generation and use of biomolecule-based information for storage. Further provided are devices-having addressable electrodes controlling polynucleotide synthesis (deprotection, extension, or cleavage, etc.) The compositions, devices, systems and methods described herein provide improved storage, density, and retrieval of biomolecule-based information.

### CROSS-REFERENCE

This application is a continuation of U.S. application Ser. No. 16/239,453, filed Jan. 3, 2019, which claims the benefit of U.S. provisional patent application No. 62/613,728 filed on Jan. 4, 2018; U.S. provisional patent application No. 62/617,067 filed on Jan. 12, 2018; and U.S. provisional patent application No. 62/650,231 filed on Mar. 29, 2018, all of which are incorporated herein by reference in their entirety.

### BACKGROUND

Biomolecule based information storage systems, e.g., DNA-based, have a large storage capacity and stability over time. However, there is a need for scalable, automated, highly accurate and highly efficient systems for generating biomolecules for information storage.

### BRIEF SUMMARY

Provided herein are devices for storing information, comprising: a solid support, wherein the solid support comprises a plurality of wells, wherein each of the wells comprises an addressable locus comprising: a synthesis surface located in a bottom region of each of the wells; a bottom electrode in addressable communication with the synthesis surface; and at least one sidewall electrode located on a sidewall of each of the wells, wherein the at least one sidewall electrode is 50 nm to 200 nm from the bottom region. Further provided herein are devices wherein the solid support comprises addressable loci at a density of at least 100×10⁶ addressable loci per cm². Further provided herein are devices wherein the solid support comprises addressable loci at a density of 100×10⁶ to 100×10⁷ addressable loci per cm². Further provided herein are devices wherein the addressable locus comprises a diameter up to about 750 nm. Further provided herein are devices wherein each of the wells comprises a depth up to about 1000 nm. Further provided herein are devices wherein each of the wells comprises a depth of 100 nm to 1000 nm. Further provided herein are devices wherein each of the wells comprises a longest cross-sectional diameter of 100 nm to 800 nm. Further provided herein are devices wherein each of the wells is cylindrical. Further provided herein are devices wherein the bottom electrode comprises a largest cross-sectional area of 10⁴ nm² to 10⁵ nm². Further provided herein are devices wherein the at least one sidewall electrode is 50 nm to 200 nm from the bottom region. Further provided herein are devices wherein the at least one sidewall electrode comprises a height of 5 nm to 25 nm. Further provided herein are devices comprising at least two sidewall electrodes. Further provided herein are devices wherein the at least one sidewall electrode and the bottom electrode are independently addressable.

Provided herein are devices for storing information, comprising: a solid support, wherein the solid support comprises a plurality of wells, wherein each of the wells comprises an addressable locus comprising: a synthesis surface located in a bottom region of each of the wells; a bottom electrode in addressable communication with the synthesis surface; at least one sidewall electrode located on a sidewall of each of the wells, wherein the synthesis surface at each addressable locus comprises at least one polynucleotide extending from the synthesis surface, and wherein the polynucleotides comprising different sequences on the solid support are present at a density of at least 100×10⁶ polynucleotides per cm². Further provided herein are devices wherein the solid support comprises polynucleotides of different sequences at a density of at least 100×10⁷ polynucleotides per cm². Further provided herein are devices wherein the solid support comprises addressable loci at a density of 100×10⁶ to 100×10⁷ polynucleotides per cm². Further provided herein are devices wherein each of the wells comprises a depth up to about 1000 nm. Further provided herein are devices wherein each of the wells comprises a depth of 100 nm to 1000 nm. Further provided herein are devices wherein the addressable locus comprises a diameter up to about 750 nm. Further provided herein are devices wherein each of the wells comprises a longest cross-sectional diameter of 100 nm to 800 nm. Further provided herein are devices wherein each of the wells is cylindrical. Further provided herein are devices wherein the bottom electrode comprises a largest cross-sectional area of 10⁴ nm² to 10⁵ nm². Further provided herein are devices wherein the at least one sidewall electrode is 50 nm to 200 nm from the bottom region. Further provided herein are devices wherein the at least one sidewall electrode comprises a height of 5 nm to 25 nm. Further provided herein are devices comprising at least two sidewall electrodes. Further provided herein are devices wherein the at least one sidewall electrode and the base electrode are independently addressable.

Provided herein are methods for storing information, comprising: (a) providing a solid support comprising a surface; (b) depositing at least one nucleoside on the surface, wherein the at least one nucleoside couples to a polynucleotide attached to the surface; and (c) repeating step b) to synthesize a plurality of polynucleotides on the surface, wherein polynucleotides having different sequences on the surface are present at a density of at least 100×10⁶ polynucleotides per cm². Further provided herein are methods wherein the density of addressable loci on the solid support is at least 100×10⁷ polynucleotides per cm². Further provided herein are methods wherein the density of addressable loci on the solid support is 100×10⁶ to 100×10⁷ polynucleotides per cm². Further provided herein are methods wherein the method further comprises cleaving at least one polynucleotide from the surface, wherein the polynucleotide is dissolved in a droplet. Further provided herein are methods wherein the method further comprises sequencing at least one polynucleotide from the surface. Further provided herein are methods wherein the nucleoside comprises a nucleoside phosphoramidite. Further provided herein are methods wherein the method further comprises drying the surface. Further provided herein are methods wherein the method further comprises washing the nucleosides away from the surface. Further provided herein are methods wherein the method further comprises a capping step. Further provided herein are methods wherein the method further comprises an oxidation step. Further provided herein are methods wherein the method further comprises a deblocking step.

Provided herein are methods for storing information, comprising: (a) providing a solid support comprising a surface; (b) depositing at droplet comprising at least one nucleoside on the surface, wherein the at least one nucleoside couples to a polynucleotide attached to the surface; and (c) repeating step b) to synthesize a plurality of polynucleotides on the surface, wherein the droplet has a volume of less than about 100 femtoliters. Further provided herein are methods wherein the droplet has a volume of less than about 50 femtoliters. Further provided herein are methods wherein the droplet has a volume of less than about 25 femtoliters to 100 femtoliters. Further provided herein are methods wherein the method further comprises cleaving at least one polynucleotide from the surface, wherein the polynucleotide is dissolved in a droplet. Further provided herein are methods wherein the method further comprises sequencing at least one polynucleotide from the surface. Further provided herein are methods wherein the nucleoside comprises a nucleoside phosphoramidite. Further provided herein are methods wherein the method further comprises drying the surface. Further provided herein are methods wherein the method further comprises washing the nucleosides away from the surface. Further provided herein are methods wherein the method further comprises a capping step. Further provided herein are methods wherein the method further comprises an oxidation step. Further provided herein are methods wherein the method further comprises a deblocking step.

Provided herein are methods for storing information, comprising: (a) providing a solid support comprising a surface; (b) depositing at least one nucleoside on the surface, wherein the at least one nucleoside couples to a polynucleotide attached to the surface; and (c) repeating step b) to synthesize a plurality of polynucleotides on the surface, wherein the time to repeat step b) using four different nucleotides is less than about 100 milliseconds. Further provided herein are methods wherein the time to repeat step b) using four different nucleotides is less than about 50 milliseconds. Further provided herein are methods wherein the time to repeat step b) using four different nucleotides is 25 milliseconds to 100 milliseconds. Further provided herein are methods wherein the method further comprises cleaving at least one polynucleotide from the surface, wherein the polynucleotide is dissolved in a droplet. Further provided herein are methods further comprising sequencing at least one polynucleotide from the surface. Further provided herein are methods wherein the nucleoside comprises a nucleoside phosphoramidite. Further provided herein are methods wherein the method further comprises drying the surface. Further provided herein are methods wherein the method further comprises washing the nucleosides away from the surface. Further provided herein are methods wherein the method further comprises a capping step. Further provided herein are methods wherein the method further comprises an oxidation step. Further provided herein are methods wherein the method further comprises a deblocking step.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary workflow for nucleic acid-based data storage.

FIG. 2 illustrates a plate configured for polynucleotide synthesis comprising 24 regions, or sub-fields, each having an array of 256 clusters.

FIG. 3 illustrates a closer view of the sub-field in FIG. 2 having 16×16 of clusters, each cluster having 121 individual loci.

FIG. 4 illustrates a detailed view of the cluster in FIG. 2, where the cluster has 121 loci.

FIG. 5A illustrates a front view of a plate with a plurality of channels.

FIG. 5B illustrates a sectional view of plate with a plurality of channels.

FIGS. 6A-6B depict a continuous loop and reel-to-reel arrangements for flexible structures.

FIGS. 6C-6D depict schemas for release and extraction of synthesized polynucleotides.

FIGS. 7A-7C depict a zoom in of a flexible structure, having spots, channels, or wells, respectively.

FIG. 8 illustrates an example of a computer system.

FIG. 9 is a block diagram illustrating architecture of a computer system.

FIG. 10 is a diagram demonstrating a network configured to incorporate a plurality of computer systems, a plurality of cell phones and personal data assistants, and Network Attached Storage (NAS).

FIG. 11 is a block diagram of a multiprocessor computer system using a shared virtual address memory space.

FIG. 12A is a front side of an example of a solid support array.

FIG. 12B is a back side of an example of a solid support array.

FIG. 13 is a schema of solid support comprising an active area and fluidics interface.

FIG. 14 is an example of rack-style instrument.

FIG. 15 depicts a solid support comprising addressable regions for nucleic acid synthesis or storage.

FIG. 16A depicts an array for synthesis using electrochemistry.

FIG. 16B depicts an array for synthesis using electrochemistry.

FIG. 17 depicts wells for nucleic acid synthesis or storage and a pitch distance between wells.

FIG. 18 illustrates an example of a solid support comprising an addressable array.

FIG. 19 illustrates an example of a solid support array, wherein the pitch approximates the length of a 240mer polynucleotide.

### DETAILED DESCRIPTION OF THE INVENTION

There is a need for larger capacity storage systems as the amount of information generated and stored is increasing exponentially. Traditional storage media have a limited capacity and require specialized technology that changes with time, requiring constant transfer of data to new media, often at a great expense. A biomolecule such as a DNA molecule provides a suitable host for information storage in-part due to its stability over time and capacity for four bit information coding, as opposed to traditional binary information coding. Thus, large amounts of data are encoded in the DNA in a relatively smaller amount of physical space than used by commercially available information storage devices. Provided herein are methods to increase DNA synthesis throughput through increased sequence density and decreased turn-around time.

### Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the polynucleotide being known and chosen before the synthesis or assembly of the nucleic acid molecules.

Provided herein are methods and compositions for production of synthetic (i.e. de novo synthesized or chemically synthesized) polynucleotides. Polynucleotides may also be referred to as oligonucleotides or oligos. Polynucleotide sequences described herein may be, unless stated otherwise, comprise DNA or RNA.

### Solid Support Based Nucleic Acid Synthesis and Storage

Described herein are devices, compositions, systems and methods for chip based nucleic acid synthesis and storage. In some instances, polynucleotides are de novo synthesized using solid support based methods as described herein. In some instances, polynucleotides are stored on a solid support following synthesis. In some instances, solid support based methods as described herein are used for storage only.

Described herein are devices, compositions, systems and methods for solid support based nucleic acid synthesis and storage, wherein one or more nucleic acid synthesizer components are integrated into a solid support. Components or functional equivalents of components may comprise temperature control units, addressable electrodes, semiconducting surfaces, fluid reservoirs, fluidics, synthesis surfaces, power sources, or other component used to synthesize polynucleotides. Any combination of integrated components is suitable for use with the devices, compositions, systems and methods described herein. In some instances, one or more components is external (non-integrated) to the solid support.

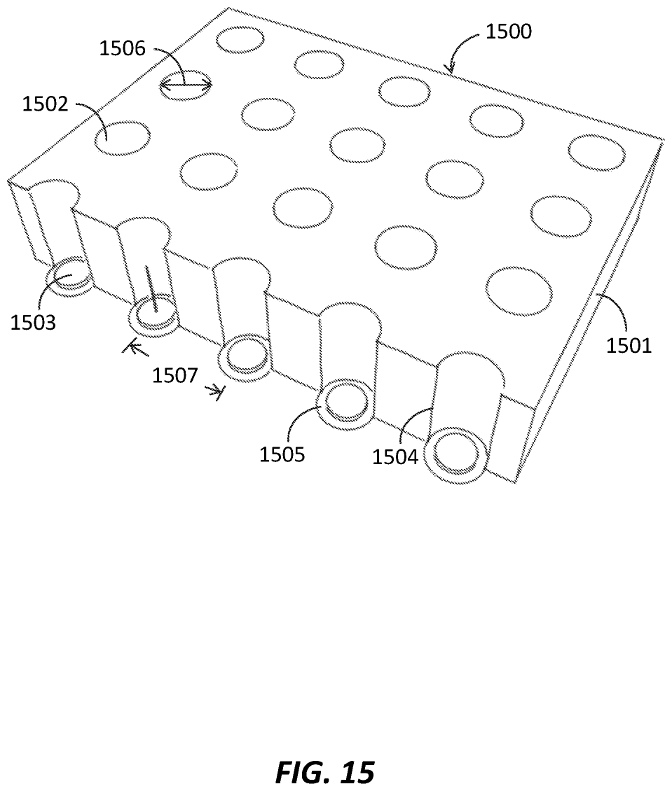

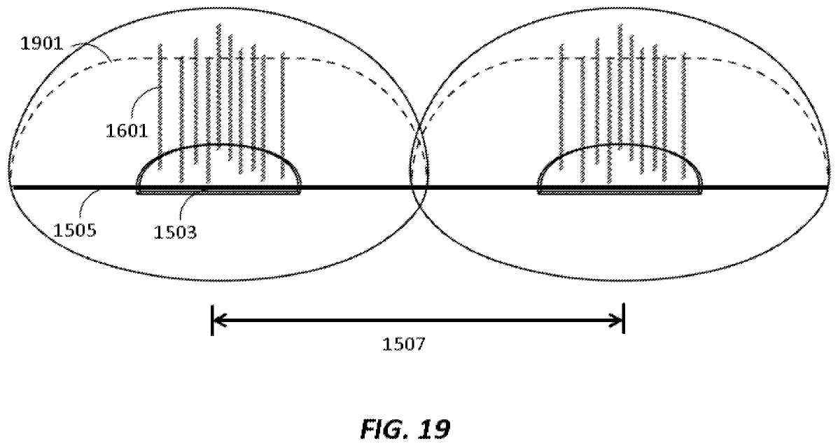

The density of unique loci for polynucleotide synthesis on a surface is often controlled by the spatial resolution achievable by reagent deposition. Additionally, reagent deposition at unique sites requires movement of either the deposition device or the receiving surface to move the site of reagent deposition from one site to another. Alternatively, coupling of bases is locally controlled at defined sites on a synthesis surface without movement of the surface or a reagent deposition device. Local control is achieved through an array of addressable electrodes 1503, wherein each electrode controls nucleoside (nucleoside phosphoramidite) coupling through electrochemistry at a specific loci on the surface 1505. In some instances, an electrode is a base electrode (or bottom electrode), located at a bottom region in addressable communication with the synthesis surface. In some instances, electrodes are sidewall electrodes, located in the side of a well. However, the conventional process of electrochemistry on a flat array in some instances is limited by the pitch distance (See FIG. 19). At high densities (e.g., short pitch distance 1507), the length of growing DNA oligos 1601 can reach from one synthesis site to the adjacent ones, mixing discrete reaction products. To avoid mixing of adjacent reaction products, the pitch distance 1507 in some embodiments is increased. Diffusion through the liquid reaction medium 1901 is an additional factor which influences spatial control of polynucleotide synthesis. Thus, it is advantageous to isolate active sites to achieve higher array densities. Nucleoside coupling is controlled by local electrodes through any number of manipulations such as generation of local chemical reagents, local removal of reagents, repulsion of reagents, restriction of solvent, attraction of solvent, or other electrochemical or physical manipulation that influences one or more steps in base coupling. In a some instances, a device 1500 comprising a plate 1501 with wells 1502 is used to synthesize polynucleotides (See FIG. 15). The bottom of each well 1505 comprises an electrode 1503 from which polynucleotides are synthesized. Each well has a cross-sectional diameter 1506, and a pitch distance between any two wells 1507. The sidewalls 1504 of the electrode in some instances comprise one or more sidewall electrodes (not shown).

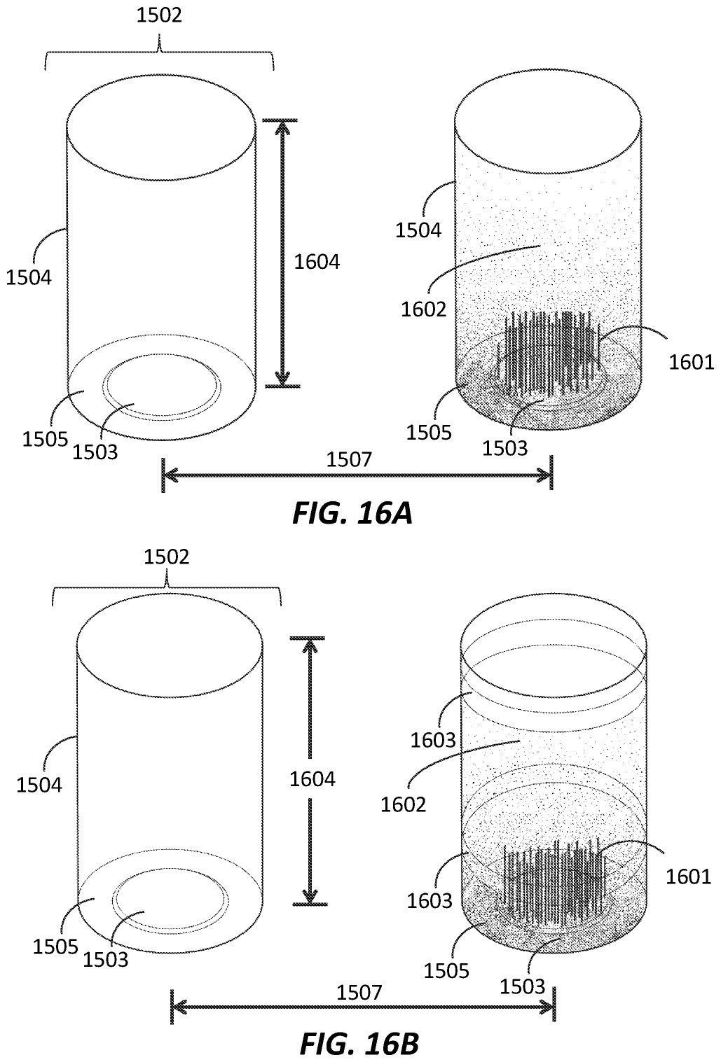

Coupling in some instances is controlled directly by control of the nucleoside addition step, a deprotection step, or other step that affects the efficiency of a nucleoside coupling reaction. In some instances, a pattern of electrodes are charged to generate a gradient of H⁺ ions 1602 on defined sites near the synthesis surface (See FIG. 16A); the polynucleotides 1601 at these sites are unblocked (wherein the polynucleotide is blocked with an acid-cleavable blocking group) and will be available for coupling to nucleosides.

Described herein are devices comprising a solid support, wherein the solid support comprises a plurality of wells, wherein each of the wells comprises an addressable locus comprising: a synthesis surface located in a bottom region of each of the wells; and at least one sidewall electrode located on a sidewall of each of the wells, wherein the electrochemical generation of reagents is spatially separated from a polynucleotide attachment point to the synthesis surface. In some instances, devices described herein further comprise a bottom electrode in addressable communication with the synthesis surface. For example, sidewall electrodes 1603 can be used to control adhesion of substrates or reagents (See FIG. 16B). In some instances, reagents comprise protons or other acid molecule. In some instances, sidewall electrodes 1603 are located at positions around the edges of the well surface (See FIG. 16B) of a well having depth 1604. In some instances, sidewall electrodes control chemical reactions occurring near the synthesis surface. For example, if acid or other reagent is generated near the synthesis surface, the portion of a polynucleotide 1601 bound to this surface will be contacted with a higher concentration of acid than the portion of the polynucleotide that is distal to the site of acid generation. This may lead to degradation of the portion of the polynucleotide which is exposed to higher concentrations of acid. Sidewall electrodes 1603 in some instances produce or control a proton gradient 1602 which results in uniform or targeted exposure of a portion of the polynucleotide 1601 to acid. Sites near uncharged electrodes do not couple with nucleosides deposited over the synthesis surface, and the pattern of charged electrodes is altered before addition of the next nucleoside. By applying a series of electrode-controlled masks to the surface, the desired polynucleotides are synthesized at exact locations on the surface. Additionally, local control of coupling in some instances reduces synthetic steps, reduces reagents/materials (due to higher polynucleotide density and reduced scale), and reduces synthesis time (no movement of the synthesis surface). Wells in some instances comprise one, two, three, four, or more than four sidewall electrodes. In some instances, wells comprise two sidewall electrodes. In some instances, each sidewall electrode is independently addressable. For example, different voltages are independently applied to two or more different sidewall electrodes. Such arrangements in some instances facilitate diffusion of reagents or polynucleotides in a defined plane between the two sidewall electrodes. Such sidewall electrodes in some instances are ring-shaped or continuous around the circumference of the well cross-section. In some instances, sidewall electrodes are discontinuous, or only partially cover a portion of a sidewall surface. For example, a sidewall electrode is continuous over about 5%, 10% 15%, 30%, 50%, 75%, or about 90% of the circumference of the well cross-section. Such sidewall electrodes in some instances have a height about equal to the well height, or about 5%, 10% 15%, 30%, 50%, 75%, or about 90% of the well height. In some instances, application of different voltages independently to two or more discontinuous sidewall electrodes causes diffusion of reagents or polynucleotides in a horizontal plane. In some instances, application of different voltages independently to two or more discontinuous sidewall electrodes causes diffusion of reagents or polynucleotides in a vertical plane.

Polynucleotide synthesis generally requires repeated deposition and removal of liquids (fluidics) on the synthesis surface. Bulk movement of fluids is some instances results in fluid loss (wetting, volume of transport lines or reaction wells), which results in low efficiency of reagent usage and higher cycle times for moving fluids. An alternative to bulk fluidics during synthesis is the use of digital fluidics, wherein reagents or reaction vessels are packaged as discrete droplets. Droplets in some instances are mixed, moved (merged, reacted), split, stored, added, removed or analyzed in discrete volumes by manipulation through a surface comprising insulated (or semiconductor-coated) electrodes, or electrowetting. Electrowetting allows for local control of fluid-surface interaction; for example energizing an electrode near a droplet results in splitting of the droplet. In some instances, droplets as described herein comprise a small volume. For example, the volume of a droplet is up to 10, 20, 50, 75, 100, 125, 150, 200, 300, 500, 800, or more than 1000 femtoliters. In some instances, the volume of a droplet is about 50 to about 200 femtoliters. In some instances, digital fluidics results in at least a 2, 3, 4, 7, 10 or more than 10× decrease in cycle times relative to bulk fluidics. In some instances, digital fluidics results in about 2× to 10× decrease in cycle times relative to bulk fluidics. In some instances, the time to complete one cycle (sequential coupling of 4 bases, including washes) is about 1, 2, 3, 5, 7, 10, 12, 15, 17, 20, 30, 50, 100, or about 200 milliseconds (ms). In some instances, the time to complete one cycle is up to 1, 2, 3, 5, 7, 10, 12, 15, 17, 20, 30, 50, 100, or up to 200 ms. In some instances, the time to complete one cycle is about 10 to about 50 ms.

Movement of fluids in or out of surfaces described herein may comprise modifications or conditions that prevent unwanted fluid movement or other phenomenon. For example, fluid movement in some instances results in the formation of bubbles or pockets of gas, which limits contact of fluids with components such as surfaces or polynucleotides. Various methods to control or minimize bubble formation are contemplated by the methods, systems, and compositions described herein. Such methods include control of fluid pressure, well geometry, or surface materials/coatings. Well geometry can be implemented to minimize bubbles. For example, tapering the well, channels, or other surface can reduce or eliminate bubble formation during fluid flow. Surface materials possessing specific wetting properties can be implemented to reduce or eliminate bubble formation. For example, surfaces described herein comprise hydrophobic materials. In some instances, surfaces described herein comprise hydrophilic materials. Pressure can be used to control bubble formation during fluid movement. Pressure in some instances is applied locally to a component, an area of a surface, a capillary/channel, or applied to an entire system. Pressure is in some instances applied either behind the direction of fluid movement, or in front of it. In some instances, back pressure is applied to prevent the formation of bubbles. Suitable pressures used for preventing bubble formation can range depending on fluid, the scale, flow geometry, and the materials used. For example, 5 to 10 atmospheres of pressure are maintained in the system. In some instances, at least 2, 3, 4, 5, 6, 7, 8, 9, 10, 11, 12, 15, 20 or more than 50 atmospheres of pressure are applied. In some instances, up to 2, 3, 4, 5, 6, 7, 8, 9, 10, 11, 12, 15, 20 or up to 50 atmospheres of pressure are applied. In some instances, about 2 to about 10, about 2 to about 8, about 2 to about 5, about 4 to about 10, about 4 to about 12, about 5 to about 15, about 5 to about 7, about 7 to about 20, about 8 to about 15, or about 10 to about 20 atmospheres of pressure are applied.

Devices described herein may utilize control units for the purpose of regulating environmental conditions, such as temperature. Temperature control units are often used to prepare or maintain conditions for storing solid supports comprising polynucleotides. Storage conditions of nucleic acids can affect their long term stability, which directly influences the quality of the digital storage information that is retrieved. Polynucleotides are optionally stored at low temperature (for example, 10 degrees C., 4 degrees C., 0 degrees C., or lower) on a solid support, wherein a temperature control unit maintains this solid support temperature. The storage medium for polynucleotides on a solid support, such as solvated or dry also influences storage stability. In some instances, polynucleotides are stored in solution, such as an aqueous solution or buffer in droplets. In some instances, polynucleotides are stored lyophilized (dry). Temperature control units in some instances increase the chip temperature to facilitate drying of polynucleotides attached thereto. Temperature control units also provide for local control of heating at addressable locations on the solid support in some instances. In some instances, following addition of the droplets comprising the polynucleotides to the solid support, the solid support is dried. In some instances, the dried solid support is later resolvated. In some instances, the solid support is stored for later use. In some instances, the solid support further comprises an index map of the polynucleotides. In some instances, the solid support further comprises metadata.

Devices described herein can comprise power sources used to energize various components of the device. Synthesis components in the solid support are optionally powered by an external power source, or a power source integrated into the solid support. Power sources may comprise batteries, solar cells, thermoelectric generators, inductive (wireless) power units, kinetic energy charger, cellular telephones, tablets, or other power source suitable for use with the synthesis components or devices described herein. In some instances, synthesis components, surfaces, or devices described herein are portable.

Fluids comprising reagents, wash solvents, or other synthesis components are deposited on the synthesis surface. Unused fluid (prior to contact with the synthesis surface) or waste fluid (after contact with the synthesis surface) is in some instances stored in one or more compartments integrated into the solid support. Alternately or in combination, polynucleotides are moved in or out of the solid support for external analysis or storage. For example, synthesized polynucleotides are cleaved from loci on the solid support in a droplet, the resulting droplet moved externally to the synthesis area of the solid support. The droplet is optionally dried for storage. In some instances, fluids are stored externally from the solid support. In some instances, a device described herein comprises a solid support with a plurality of fluidics ports which allow movement of fluids in and out of the solid support. In some instances, ports are oriented on the sides of the solid support, by other configurations are also suitable for delivery of fluids to the synthesis surface. Such a device often comprises, for example, at least 1, 2, 5, 10, 20, 50, 100, 200, 500, 1000, 2000, 5000, or at least 10,000 ports per mm length of a solid support. In some instances, a device described herein comprises about 100 to about 5000 ports per mm per length of a solid support.

Described herein are addressable electrodes integrated into a solid support. Electrodes comprise without limitation conductors, insulators, or semi-conductors, and are fabricated of materials well known in the art. Materials may comprise metals, non-metals, mixed-metal oxides, nitrides, carbides, silicon-based materials, or other material. In some instances, metal oxides include TiO₂, Ta₂O₅, Nb₂O₅, Al₂O₃, BaO, Y₂O₃, HfO₂, SrO or other metal oxide known in the art. In some instances, metal carbides include TiC, WC, ThC₂, ThC, VC, W₂C, ZrC, HfC, NbC, TaC, Ta₂C, or other metal carbide known in the art. In some instances, metal nitrides include GaN, InN, BN, Be₃N₂, Cr₂N, MoN, Si₃N₄, TaN, Th₂N₂, VN, ZrN, TiN, HfN, NbC, WN, TaN, or other metal nitride known in the art. In some instances, a device disclosed herein is manufactured with a combination of materials listed herein or any other suitable material known in the art. Electrodes can possess any shape, including discs, rods, wells, posts, a substantially planar shape, or any other form suited for nucleic acid synthesis. The or cross-sectional area of each electrode varies as a function of the size of the loci for polynucleotide synthesis, but in some instances is up to 500 um², 200 um², 100 um², 75 um², 50 um², 25 um², 10 um², less than 5 um². In some instances, the cross-sectional area of each electrode is about 500 um² to 10 um², about 100 um² to 25 um², or about 150 um² to 50 um². In some instances, the cross-sectional area of each electrode is about 150 um² to 50 um². Devices provide herein include electrodes having a diameter that varies as a function of the size of the loci for polynucleotide synthesis. Exemplary electrode diameters include, without limitation, up to 500 um, 200 um, 100 um, 75 um, 50 um, 25 um, 10 um, less than 5 um. In some instances, the diameter of each electrode is about 500 um to 10 um, about 100 um to 25 um, about 100 um to about 200 um, about 50 um to about 200 um, or about 150 um to 50 um. In some instances, the diameter of each electrode is about 200 um to 50 um. In some instances, the diameter of each electrode is about 200 um to 100 um. In some instances, the diameter of each electrode is up to 500 nm, 200 nm, 100 nm, 75 nm, 50 nm, 25 nm, 10 nm, less than 5 nm. In some instances, the diameter of each electrode is about 500 nm to 10 nm, about 100 nm to 25 nm, about 100 nm to about 200 nm, about 50 nm to about 200 nm, or about 150 nm to 50 nm. In some instances, the diameter of each electrode is about 200 nm to 50 nm. In some instances, the diameter of each electrode is about 200 nm to 100 nm. The thickness of each electrode varies as a function of the size of the loci for polynucleotide synthesis, but in some instances is about 50 nm, 100 nm, 200 nm, 500 nm, 750 nm, 1000 nm, 1200 nm, 1500 nm, 2000 nm, 2500 nm, 3000 nm, or about 3500 nm. In some instances the thickness of the electrode is at least 50 nm, 100 nm, 200 nm, 500 nm, 750 nm, 1000 nm, 1200 nm, 1500 nm, 2000 nm, 2500 nm, 3000 nm, or at least 3500 nm. In some instances the thickness of the electrode is at least 1 um, 2 um, 3 um, 5 um, 10 um, 15 um, 20 um, 30 um, 50 um or at least 75 um. In some instances the thickness of the electrode is about 1 um, 2 um, 3 um, 5 um, 10 um, 15 um, 20 um, 30 um, 50 um or about 75 um. In some instances the thickness of the electrode is up to 1 um, 2 um, 3 um, 5 um, 10 um, 15 um, 20 um, 30 um, 50 um or up to 75 um. In some instances the thickness of the electrode is up to 50 nm, 100 nm, 200 nm, 500 nm, 750 nm, 1000 nm, 1200 nm, 1500 nm, 2000 nm, 2500 nm, 3000 nm, or up to 3500 nm. In some instances the thickness of the electrode is about 20 nm to 3000 nm, about 50 nm to 2500, about 100 nm to 750 nm, about 400 nm to 750 nm, about 500 nm to 3000 nm, or about 1000 nm to 3000 nm. In some instances the thickness of the electrode is about 10 um to about 20 um. In some instances the thickness of the electrode is about 5 um to about 50 um, about 10 um to about 30 um, about 15 um to about 25 um, or about 30 um to about 50 um. In some instances, electrodes are coated with additional materials such as semiconductors or insulators. In some instances, electrodes are coated with materials for polynucleotide attachment and synthesis. The size, shape, pattern, or orientation of electrodes is in some instances chosen to minimize deleterious side reactions caused by electrochemically generated reagents. In some instances combinations of electrodes are used, such as a grid of addressable electrodes and a common electrode. Electrodes are in some instances cathodes or anodes. Electrodes or arrays of electrodes can be positioned anywhere on or near the polynucleotide surface. In some instances, electrodes are placed on the bottom of loci for synthesis, such as the bottom of a well or channel. In some instances, electrodes are placed in the sidewalls of a well or channel (“sidewall electrodes”). A plurality of sidewall electrodes are in some instances present on the sides of a well. Electrodes positioned at different locations in the device can have different functions, and are independently or simultaneously addressable. In some instances, an electrode in the bottom of a well is used to cleave polynucleotides from the surface at one or more loci, and a sidewall electrode is used to generate acid to deprotect polynucleotides. In some instances, an electrode in the bottom of a well is used to cleave polynucleotides from the surface at one or more loci, and a first sidewall electrode is used to generate acid to deprotect polynucleotides, and a second sidewall electrode is used to move polynucleotides out of the well after cleavage. In exemplary configurations, sidewall electrodes are located about 10 nm, about 25 nm, about 50 nm, about 75 nm, about 100 nm, about 125 nm, or about 200 nm above the synthesis surface. In some instances, sidewall electrodes are located about 10 nm to about 100 nm, about 50 nm to about 150 nm, about 40 nm to 100 nm, about 75 nm to about 125 nm, about 100 to 300 nm above the synthesis surface. In some instances, multiple sidewall electrodes are located at different heights above the synthesis surface. For example, a locus comprises at least one sidewall electrode, at least 2 sidewall electrodes, at least 3 sidewall electrodes, or more than 3 sidewall electrodes. In exemplary configurations, sidewall electrodes have a height of about 1 nm, about 5 nm, about 10 nm, about 20 nm, about 25 nm about 30 nm, about 40 nm, or about 50 nm. In some instances, sidewall electrodes have a height of 1 nm to 20 nm, 2 nm to 30 nm, 5 nm to 20 nm, 10 nm to 40 nm, or 5 nm to 25 nm.

Electrode surfaces can support the movement, conformation, synthesis, growth, and release of polynucleotides. In some instances, electrodes are coated with one or more layers. In some instances, the layer is a monolayer which facilities attachment of a linker. In some instances, the electrode is charged to influence the area of the monolayer to be functionalized with a linker. This allows for masking of specific areas for chemical functionalization, such as modifying the surface with hydrophobic or hydrophilic chemical groups. In some instances, the electrode is charged to influence the area of the monolayer to be extended with a nucleoside monomer. This in some instances includes generation of reagents to facilitate or prevent coupling of monomers to synthesis surfaces in the vicinity of an electrode. In some instances, the electrode is charged to influence the area of the monolayer which releases polynucleotides. Such controlled release of specific polynucleotides in a specific order in some instances is used to control the assembly of synthesized monomers into larger polynucleotides. For example, an iterative polynucleotide assembly process is optimized by exploring which various combinations of polynucleotides that are released and allowed to hybridize for overlap PCR assembly.

Each electrode can control one, or a plurality of different loci for synthesis, wherein each locus for synthesis has a density of polynucleotides. In some instances, the density is at least 1 oligo per 10 nm², 20, 50, 100, 200, 500, 1,000, 2,000, 5,000 or at least 1 oligo per 10,000 nm². In some instances, the density is about 1 oligo per 10 nm² to about 1 oligo per 5,000 nm², about 1 oligo per 50 nm² to about 1 oligo per 500 nm², or about 1 oligo per 25 nm² to about 1 oligo per 75 nm². In some instances, the density of polynucleotides is about 1 oligo per 25 nm² to about 1 oligo per 75 nm².

Provided herein are devices for polynucleotide synthesis having various types of electrodes. In some instances, the device comprises a reference electrode. Reference electrodes are placed near the synthesis surface or in the case of a well or channel, for example, above a well or channel. In some instances, a reference electrode is about 1 to about 50 um above the synthesis surface, about 2 um to about 40 um, about 3 um to about 30 um, about 5 um to about 20 um, about 10 to about 20 um, about 15 to about 50 um, about 30 to about 50 um, about 5 um to about 30 um or about 7 um to about 25 um. In some instances a reference electrode is about 1, 2, 5, 7, 8, 9, 10, 12, 14, 16, 18, 20, 22, 24, 26 um or more than 26 um above the synthesis surface. In some instances a reference electrode is up to 1, 2, 5, 7, 8, 9, 10, 12, 14, 16, 18, 20, 22, 24, 26 um or up to 26 um above the synthesis surface. In some instances a reference electrode is at least 1, 2, 5, 7, 8, 9, 10, 12, 14, 16, 18, 20, 22, 24, 26 um or at least 26 um above the synthesis surface. Reference electrodes in some instances are adjacent to synthesis surfaces, such as adjacent to a well or channel. Each locus in some instances has one corresponding reference electrode. In some instances, each locus shares a common reference electrode with one or more adjacent loci. The devices described herein may comprise any number of reference electrodes. In some instances the reference electrode is a single, uniform plate. In some instances, devices comprise a plurality of reference electrodes. A reference electrode can address a plurality of loci, for example 2, 3, 4, 5, 10 or more loci.

Described herein are devices, compositions, systems and methods for solid support based nucleic acid synthesis and storage, wherein the solid support has varying dimensions. In some instances, a size of the solid support is between about 40 and 120 mm by between about 25 and 100 mm. In some instances, a size of the solid support is about 80 mm by about 50 mm. In some instances, a width of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, a height of a solid support is at least or about 10 mm, 20 mm, 40 mm, 60 mm, 80 mm, 100 mm, 150 mm, 200 mm, 300 mm, 400 mm, 500 mm, or more than 500 mm. In some instances, the solid support has a planar surface area of at least or about 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness of the solid support is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness of the solid support include 275 mm, 375 mm, 525 mm, 625 mm, 675 mm, 725 mm, 775 mm and 925 mm. In some instances, the thickness of the solid support is at least or about 0.5 mm, 1.0 mm, 1.5 mm, 2.0 mm, 2.5 mm, 3.0 mm, 3.5 mm, 4.0 mm, or more than 4.0 mm.

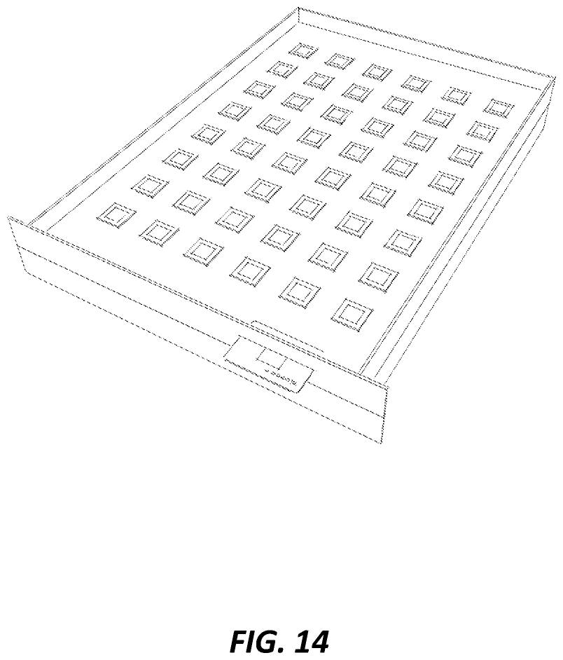

Described herein are devices wherein two or more solid supports are assembled. In some instances, solid supports are interfaced together on a larger unit. Interfacing may comprise exchange of fluids, electrical signals, or other medium of exchange between solid supports. This unit is capable of interface with any number of servers, computers, or networked devices. For example, a plurality of solid support is integrated onto a rack unit, which is conveniently inserted or removed from a server rack. The rack unit may comprise any number of solid supports. In some instances the rack unit comprises at least 1, 2, 5, 10, 20, 50, 100, 200, 500, 1000, 2000, 5000, 10,000, 20,000, 50,000, 100,000 or more than 100,000 solid supports. In some instances, two or more solid supports are not interfaced with each other. Nucleic acids (and the information stored in them) present on solid supports can be accessed from the rack unit. See e.g., FIG. 14. Access includes removal of polynucleotides from solid supports, direct analysis of polynucleotides on the solid support, or any other method which allows the information stored in the nucleic acids to be manipulated or identified. Information in some instances is accessed from a plurality of racks, a single rack, a single solid support in a rack, a portion of the solid support, or a single locus on a solid support. In various instances, access comprises interfacing nucleic acids with additional devices such as mass spectrometers, HPLC, sequencing instruments, PCR thermocyclers, or other device for manipulating nucleic acids. Access to nucleic acid information in some instances is achieved by cleavage of polynucleotides from all or a portion of a solid support. Cleavage in some instances comprises exposure to chemical reagents (ammonia or other reagent), electrical potential, radiation, heat, light, acoustics, or other form of energy capable of manipulating chemical bonds. In some instances, cleavage occurs by charging one or more electrodes in the vicinity of the polynucleotides. In some instances, electromagnetic radiation in the form of UV light is used for cleavage of polynucleotides. In some instances, a lamp is used for cleavage of polynucleotides, and a mask mediates exposure locations of the UV light to the surface. In some instances, a laser is used for cleavage of polynucleotides, and a shutter opened/closed state controls exposure of the UV light to the surface. In some instances, access to nucleic acid information (including removal/addition of racks, solid supports, reagents, nucleic acids, or other component) is completely automated.

Solid supports as described herein comprise an active area. In some instances, the active area comprises addressable regions or loci for nucleic acid synthesis. In some instances, the active area comprises addressable regions or loci for nucleic acid storage.

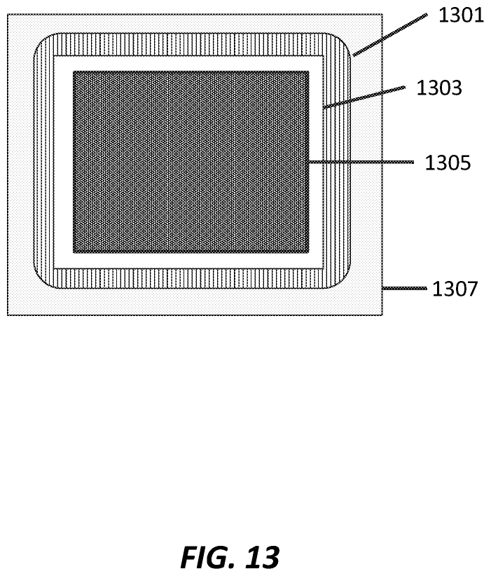

The active area comprises varying dimensions. For example, the dimension of the active area is between about 1 mm to about 50 mm by about 1 mm to about 50 mm. In some instances, the active area comprises a width of at least or about 0.5, 1, 1.5, 2, 2.5, 3, 5, 5, 10, 12, 14, 16, 18, 20, 25, 30, 35, 40, 45, 50, 60, 70, 80, or more than 80 mm. In some instances, the active area comprises a height of at least or about 0.5, 1, 1.5, 2, 2.5, 3, 5, 5, 10, 12, 14, 16, 18, 20, 25, 30, 35, 40, 45, 50, 60, 70, 80, or more than 80 mm. An exemplary active area within a solid support is seen in FIG. 13. A package 1307 comprises an active area 1305 within a solid support 1303. The package 1307 also comprises a fluidics interface 1301.

Described herein are devices, compositions, systems and methods for solid support based nucleic acid synthesis and storage, wherein the solid support has a number of sites (e.g., spots) or positions for synthesis or storage. In some instances, the solid support comprises up to or about 10,000 by 10,000 positions in an area. In some instances, the solid support comprises between about 1000 and 20,000 by between about 1000 and 20,000 positions in an area. In some instances, the solid support comprises at least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions by least or about 10, 30, 50, 75, 100, 200, 300, 400, 500, 1000, 2000, 3000, 4000, 5000, 6000, 7000, 8000, 9000, 10,000, 12,000, 14,000, 16,000, 18,000, 20,000 positions in an area. In some instances the area is up to 0.25, 0.5, 0.75, 1.0, 1.25, 1.5, or 2.0 inches squared. In some instances, the solid support comprises addressable loci having a pitch of at least or about 0.1, 0.2, 0.25, 0.3, 0.4, 0.5, 1.0, 1.5, 2.0, 2.5, 3.0, 3.5, 4.0, 4.5, 5, 6, 7, 8, 9, 10, or more than 10 um. In some instances, the solid support comprises addressable loci having a pitch of about 5 um. In some instances, the solid support comprises addressable loci having a pitch of about 2 um. In some instances, the solid support comprises addressable loci having a pitch of about 1 um. In some instances, the solid support comprises addressable loci having a pitch of about 0.2 um. In some instances, the solid support comprises addressable loci having a pitch of about 0.2 um to about 10 um, about 0.2 to about 8 um, about 0.5 to about 10 um, about 1 um to about 10 um, about 2 um to about 8 um, about 3 um to about 5 um, about 1 um to about 3 um or about 0.5 um to about 3 um. In some instances, the solid support comprises addressable loci having a pitch of about 0.1 um to about 3 um. See e.g. FIG. 15, FIG. 16A, and FIG. 16B.

The solid support for nucleic acid synthesis or storage as described herein comprises a high capacity for storage of data. For example, the capacity of the solid support is at least or about 1, 2, 3, 4, 5, 6, 7, 8, 9, 10, 20, 50, 100, 200, 300, 400, 500, 600, 700, 800, 900, 1000, or more than 1000 petabytes. In some instances, the capacity of the solid support is between about 1 to about 10 petabytes or between about 1 to about 100 petabytes. In some instances, the capacity of the solid support is about 100 petabytes. In some instances, the data is stored as addressable arrays of packets as droplets. In some instances, the data is stored as addressable arrays of packets as droplets on a spot. In some instances, the data is stored as addressable arrays of packets as dry wells. In some instances, the addressable arrays comprise at least or about 1, 2, 3, 4, 5, 6, 7, 8, 9, 10, 20, 50, 100, 200, or more than 200 gigabytes of data. In some instances, the addressable arrays comprise at least or about 1, 2, 3, 4, 5, 6, 7, 8, 9, 10, 20, 50, 100, 200, or more than 200 terabytes of data. In some instances, an item of information is stored in a background of data. For example, an item of information encodes for about 10 to about 100 megabytes of data and is stored in 1 petabyte of background data. In some instances, an item of information encodes for at least or about 1, 10, 20, 30, 40, 50, 60, 70, 80, 90, 100, 150, 200, 300, 400, 500, or more than 500 megabytes of data and is stored in 1, 10, 20, 30, 40, 50, 60, 70, 80, 90, 100, 150, 200, 300, 400, 500, or more than 500 petabytes of background data.

Provided herein are devices, compositions, systems and methods for solid support based nucleic acid synthesis and storage, wherein following synthesis, the polynucleotides are collected in packets as one or more droplets. In some instances, the polynucleotides are collected in packets as one or more droplets and stored. In some instances, a number of droplets is at least or about 1, 10, 20, 50, 100, 200, 300, 500, 1000, 2500, 5000, 75000, 10,000, 25,000, 50,000, 75,000, 100,000, 1 million, 5 million, 10 million, 25 million, 50 million, 75 million, 100 million, 250 million, 500 million, 750 million, or more than 750 million droplets. In some instances, a droplet volume comprises 5, 10, 15, 20, 25, 30, 35, 40, 45, 50, 55, 60, 65, 70, 75, 80, 85, 90, 95, 100, or more than 100 um (micrometer) in diameter. In some instances, a droplet volume comprises 1-100 um, 10-90 um, 20-80 um, 30-70 um, or 40-50 um in diameter.

In some instances, the polynucleotides that are collected in the packets comprise a similar sequence. In some instances, the polynucleotides further comprise a non-identical sequence to be used as a tag or barcode. For example, the non-identical sequence is used to index the polynucleotides stored on the solid support and to later search for specific polynucleotides based on the non-identical sequence. Exemplary tag or barcode lengths include barcode sequences comprising, without limitation, about 1, 2, 3, 4, 5, 6, 7, 8, 9, 10, 15, 20, 25 or more bases in length. In some instances, the tag or barcode comprise at least or about 10, 50, 75, 100, 200, 300, 400, or more than 400 base pairs in length.

Provided herein are devices, compositions, systems and methods for solid support based nucleic acid synthesis and storage, wherein the polynucleotides are collected in packets comprising redundancy. For example, the packets comprise about 100 to about 1000 copies of each polynucleotide. In some instances, the packets comprise at least or about 50, 75, 100, 200, 300, 400, 500, 600, 700, 800, 900, 1000, 1200, 1400, 1600, 1800, 2000, or more than 2000 copies of each polynucleotide. In some instances, the packets comprise about 1000× to about 5000× synthesis redundancy. Synthesis redundancy in some instances is at least or about 500×, 1000×, 1500×, 2000×, 2500×, 3000×, 3500×, 4000×, 5000×, 6000×, 7000×, 8000×, or more than 8000×. The polynucleotides that are synthesized using solid support based methods as described herein comprise various lengths. In some instances, the polynucleotides are synthesized and further stored on the solid support. In some instances, the polynucleotide length is in between about 100 to about 1000 bases. In some instances, the polynucleotides comprise at least or about 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more than 2000 bases in length.

### Nucleic Acid Based Information Storage

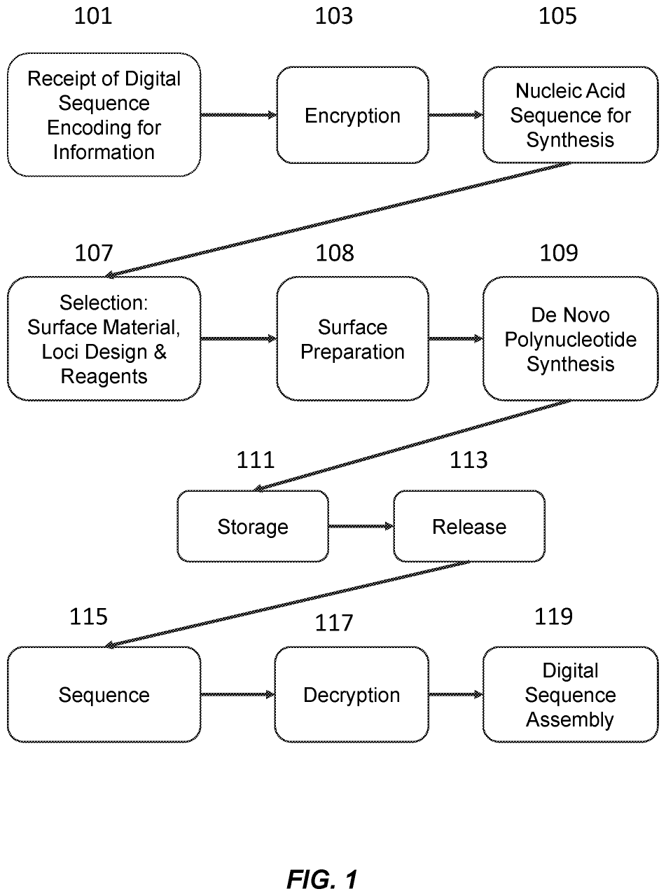

Provided herein are devices, compositions, systems and methods for nucleic acid-based information (data) storage. An exemplary workflow is provided in FIG. 1. In a first step, a digital sequence encoding an item of information (i.e., digital information in a binary code for processing by a computer) is received 101. An encryption 103 scheme is applied to convert the digital sequence from a binary code to a nucleic acid sequence 105. A surface material for nucleic acid extension, a design for loci for nucleic acid extension (aka, arrangement spots), and reagents for nucleic acid synthesis are selected 107. The surface of a structure is prepared for nucleic acid synthesis 108. De novo polynucleotide synthesis is performed 109. The synthesized polynucleotides are stored 111 and available for subsequent release 113, in whole or in part. Once released, the polynucleotides, in whole or in part, are sequenced 115, subject to decryption 117 to convert nucleic sequence back to digital sequence. The digital sequence is then assembled 119 to obtain an alignment encoding for the original item of information.

### Items of Information

Optionally, an early step of data storage process disclosed herein includes obtaining or receiving one or more items of information in the form of an initial code. Items of information include, without limitation, text, audio and visual information. Exemplary sources for items of information include, without limitation, books, periodicals, electronic databases, medical records, letters, forms, voice recordings, animal recordings, biological profiles, broadcasts, films, short videos, emails, bookkeeping phone logs, internet activity logs, drawings, paintings, prints, photographs, pixelated graphics, and software code. Exemplary biological profile sources for items of information include, without limitation, gene libraries, genomes, gene expression data, and protein activity data. Exemplary formats for items of information include, without limitation, .txt, .PDF, .doc, .docx, .ppt, .pptx, .xls, .xlsx, .rtf, .jpg, gif, .psd, .bmp, .tiff, .png, and .mpeg. The amount of individual file sizes encoding for an item of information, or a plurality of files encoding for items of information, in digital format include, without limitation, up to 1024 bytes (equal to 1 KB), 1024 KB (equal to 1 MB), 1024 MB (equal to 1 GB), 1024 GB (equal to 1 TB), 1024 TB (equal to 1PB), 1 exabyte, 1 zettabyte, 1 yottabyte, 1 xenottabyte or more. In some instances, an amount of digital information is at least 1 gigabyte (GB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 gigabytes. In some instances, the amount of digital information is at least 1 terabyte (TB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 terabytes. In some instances, the amount of digital information is at least 1 petabyte (PB). In some instances, the amount of digital information is at least 1, 2, 3, 4, 5, 6, 7, 8, 9, 10, 20, 50, 100, 200, 300, 400, 500, 600, 700, 800, 900, 1000 or more than 1000 petabytes.

### Structures for Polynucleotide Synthesis

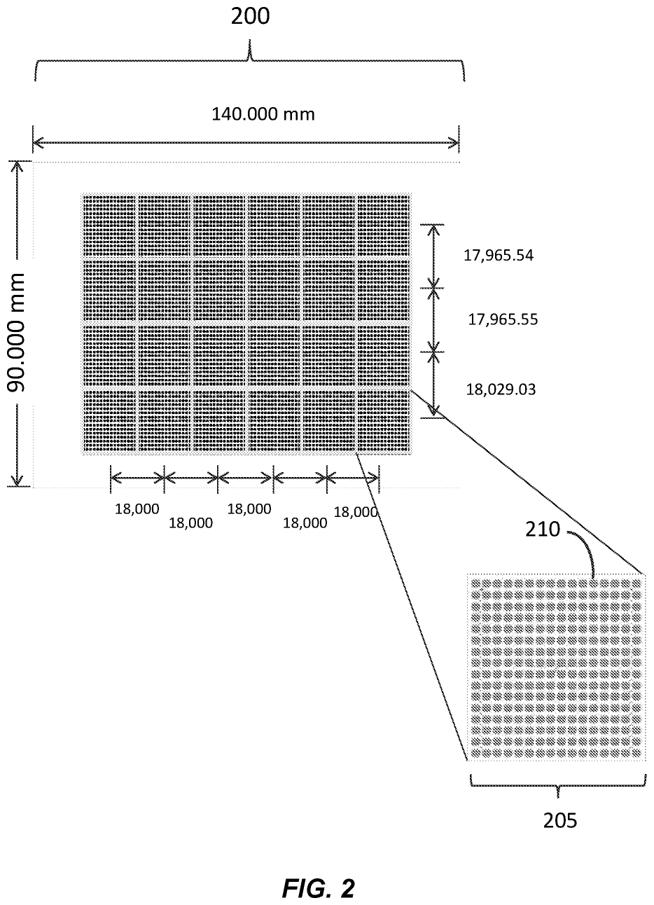

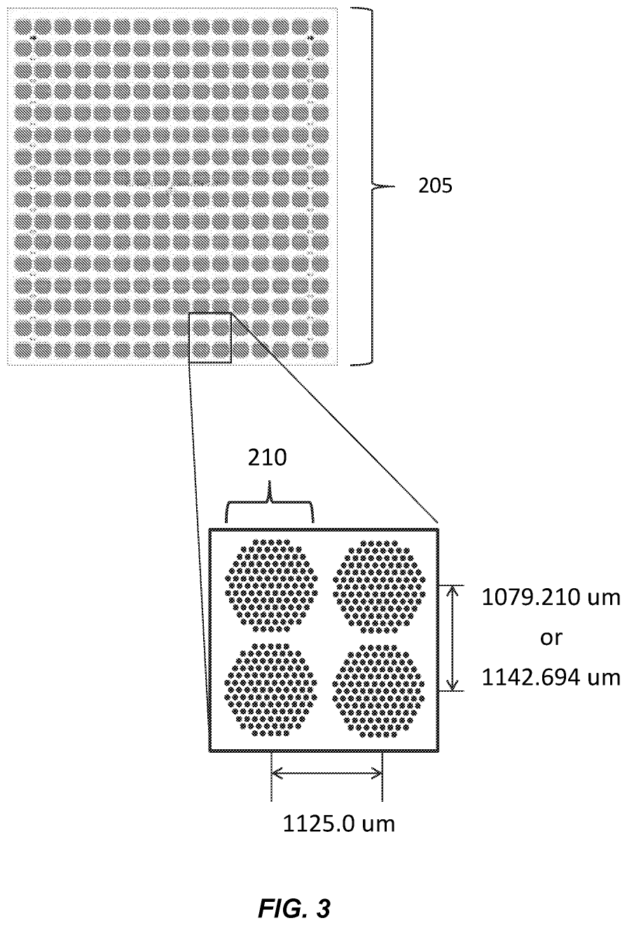

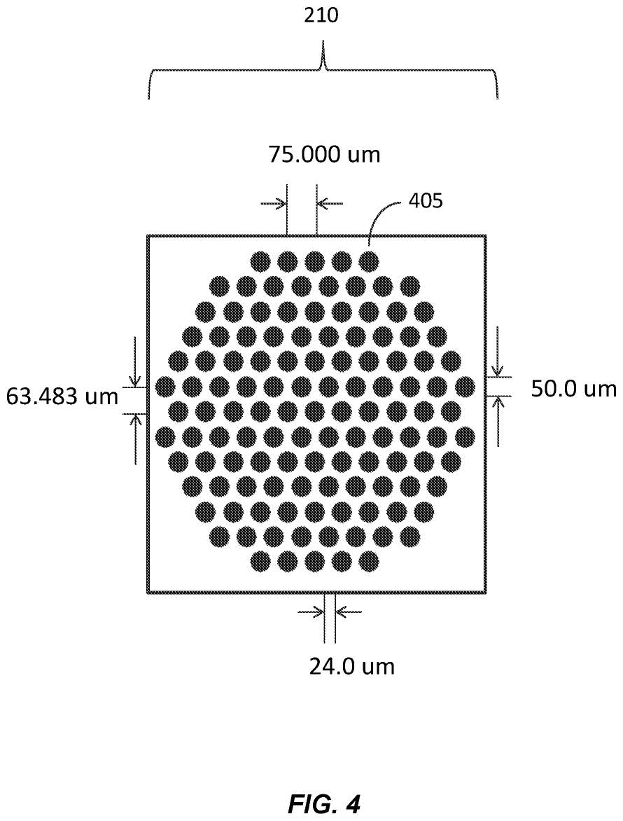

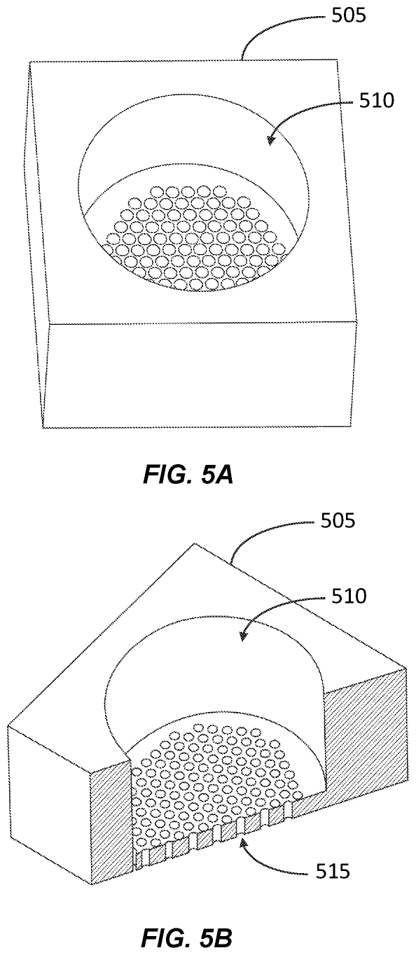

Provided herein are rigid or flexibles structures for polynucleotide synthesis. In the case of rigid structures, provided herein are devices having a structure for the generation of a library of polynucleotides. In some instances, the structure comprises a plate. An exemplary structure 200 is illustrated in FIG. 2, wherein the structure 200 has about the same size dimensions as a standard 96 well plate: 140 mm by 90 mm. The structure 200 comprises clusters grouped in 24 regions or sub-fields 205, each sub-field 205 comprising an array of 256 clusters 210. An expanded view of an exemplary sub-field 205 is shown in FIG. 3. In the expanded view of four clusters (FIG. 3), a single cluster 210, has a Y axis cluster pitch (distance from center to center of adjacent clusters) of 1079.210 um or 1142.694 um, and an X axis cluster pitch of 1125 um. An illustrative cluster 210 is depicted in FIG. 4, where the Y axis loci pitch (distance from center to center of adjacent loci) is 63.483 um, and an X axis loci pitch is 75 um. The locus width at the longest part, e.g., diameter for a circular locus, is 50 um and the distance between loci is 24 um. The number of loci 405 in the exemplary cluster in FIG. 4 is 121. The loci may be flat, wells, or channels. An exemplary channel arrangement is illustrated in FIGS. 5A-5B where a plate 505 is illustrated comprising a main channel 510 and a plurality of channels 515 connected to the main channel 510. The connection between the main channel 510 and the plurality of channels 515 provides for a fluid communication for flow paths from the main channel 510 to the each of the plurality of channels 515. A plate 505 described herein can comprise multiple main channels 510. The plurality of channels 515 collectively forms a cluster within the main channel 510.

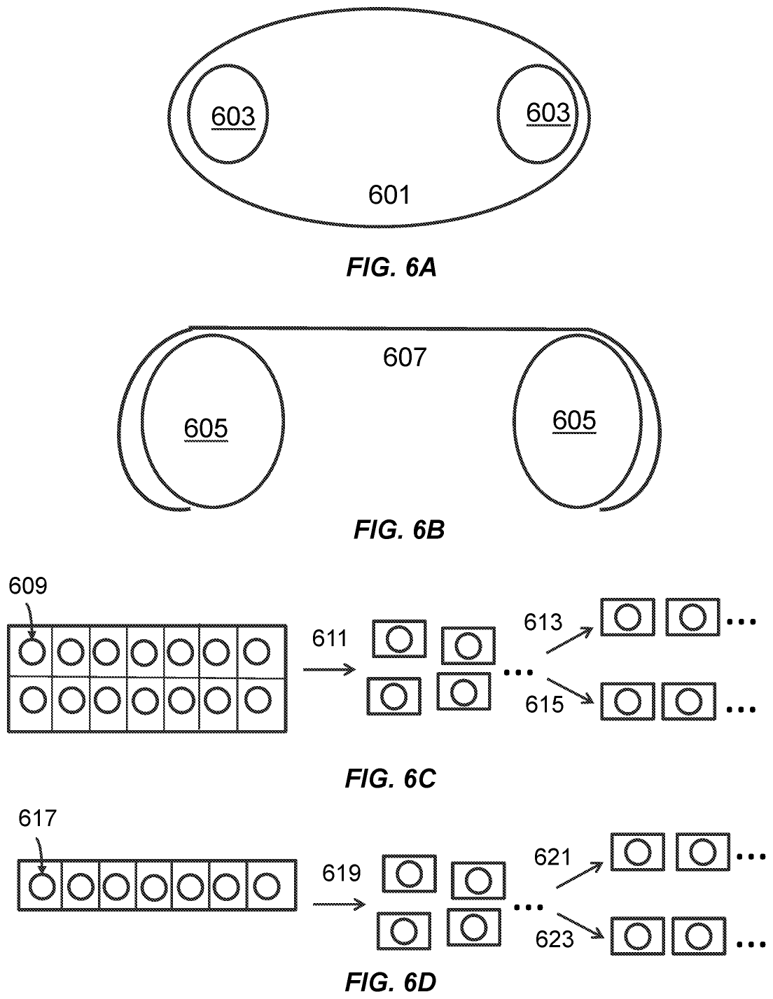

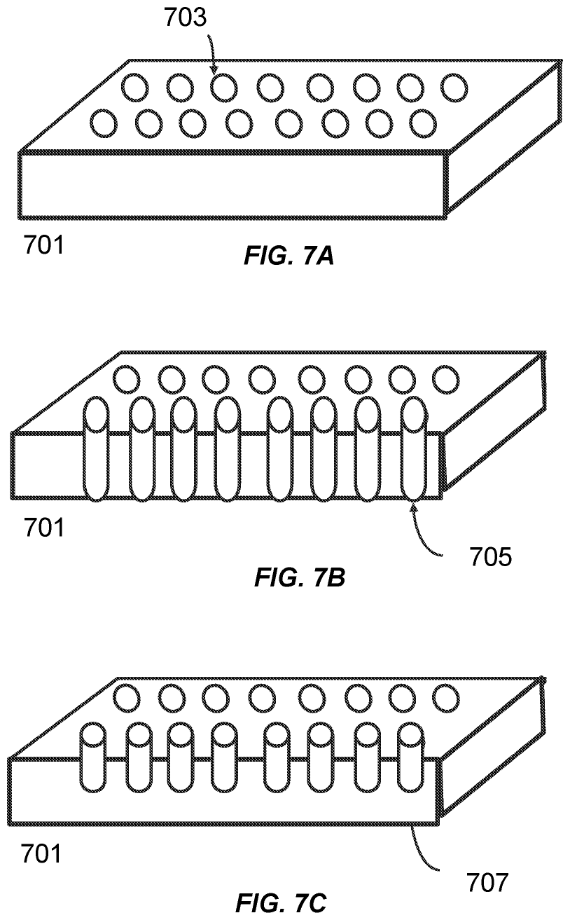

In the case of flexible structures, provided herein are devices wherein the flexible structure comprises a continuous loop 601 wrapped around one or more fixed structures, e.g., a pair of rollers 603 or a non-continuous flexible structure 607 wrapped around separate fixed structures, e.g., a pair reels 605. See FIGS. 6A-6B. In some instances, the structures comprise multiple regions for polynucleotide synthesis. An exemplary structure is illustrated in FIG. 6C where a plate comprises distinct regions 609 for polynucleotide synthesis. The distinct regions 609 may be separated 611 by breaking or cutting. Each of the distinct regions may be further released, sequenced, decrypted, and read 613 or stored 615. An alternative structure is illustrated in FIG. 6D in which a tape comprises distinct regions 617 for polynucleotide synthesis. The distinct regions 617 may be separated 619 by breaking or cutting. Each of the distinct regions may be further released, sequenced, decrypted, and read 621 or stored 623. Provided herein are flexible structures having a surface with a plurality of loci for polynucleotide extension. FIGS. 7A-7C show a zoom in of the locus in the flexible structure. Each locus in a portion of the flexible structure 701, may be a substantially planar spot 703 (e.g., flat), a channel 705, or a well 707. In some instances, each locus of the structure has a width of about 10 um and a distance between the center of each structure of about 21 um. Loci may comprise, without limitation, circular, rectangular, tapered, or rounded shapes. Alternatively or in combination, the structures are rigid. In some instances, the rigid structures comprise loci for polynucleotide synthesis. In some instances, the rigid structures comprise substantially planar regions, channels, or wells for polynucleotide synthesis.

In some instances, a well described herein has a width to depth (or height) ratio of 1 to 0.01, wherein the width is a measurement of the width at the narrowest segment of the well. In some instances, a well described herein has a width to depth (or height) ratio of 0.5 to 0.01, wherein the width is a measurement of the width at the narrowest segment of the well. In some instances, a well described herein has a width to depth (or height) ratio of about 0.01, 0.05, 0.1, 0.15, 0.16, 0.2, 0.5, or 1. Provided herein are structures for polynucleotide synthesis comprising a plurality of discrete loci for polynucleotide synthesis. Exemplary structures for the loci include, without limitation, substantially planar regions, channels, wells or protrusions. Structures described herein are may comprise a plurality of clusters, each cluster comprising a plurality of wells, loci or channels. Alternatively, described herein are may comprise a homogenous arrangement of wells, loci or channels. Structures provided herein may comprise wells having a height or depth from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a well is less than 100 um, less than 80 um, less than 60 um, less than 40 um or less than 20 um. In some instances, well height is about 10, 20, 30, 40, 50, 60, 70, 80, 90, 100, 200, 300, 400, 500 um or more. In some instances, the height or depth of the well is at least 10, 25, 50, 75, 100, 200, 300, 400, 500, 600, 700, 800, 900, 1000, or more than 1000 nm. In some instances, the height or depth of the well is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the height or depth of the well is in a range of about 50 nm to about 1 um. In some instances, well height is about 10, 20, 30, 40, 50, 60, 70, 80, 90, 100, 200, 300, 400, 500, 700, 800, 900 or about 1000 nm.

Structures for polynucleotide synthesis provided herein may comprise channels. The channels may have a width to depth (or height) ratio of 1 to 0.01, wherein the width is a measurement of the width at the narrowest segment of the microchannel. In some instances, a channel described herein has a width to depth (or height) ratio of 0.5 to 0.01, wherein the width is a measurement of the width at the narrowest segment of the microchannel. In some instances, a channel described herein has a width to depth (or height) ratio of about 0.01, 0.05, 0.1, 0.15, 0.16, 0.2, 0.5, or 1.

Described herein are structures for polynucleotide synthesis comprising a plurality of discrete loci. Structures comprise, without limitation, substantially planar regions, channels, protrusions, or wells for polynucleotide synthesis. In some instances, structures described herein are provided comprising a plurality of channels, wherein the height or depth of the channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some cases, the height of a channel is less than 100 um, less than 80 um, less than 60 um, less than 40 um or less than 20 um. In some cases, channel height is about 10, 20, 30, 40, 50, 60, 70, 80, 90, 100, 200, 300, 400, 500 um or more. In some instances, the height or depth of the channel is at least 10, 25, 50, 75, 100, 200, 300, 400, 500, 600, 700, 800, 900, 1000, or more than 1000 nm. In some instances, the height or depth of the channel is in a range of about 10 nm to about 1000 nm, about 25 nm to about 900 nm, about 50 nm to about 800 nm, about 75 nm to about 700 nm, about 100 nm to about 600 nm, or about 200 nm to about 500. Channels described herein may be arranged on a surface in clusters or as a homogenous field.

The width of a locus on the surface of a structure for polynucleotide synthesis described herein may be from about 0.1 um to about 500 um, from about 0.5 um to about 500 um, from about 1 um to about 200 um, from about 1 um to about 100 um, from about 5 um to about 100 um, or from about 0.1 um to about 100 um, for example, about 90 um, 80 um, 70 um, 60 um, 50 um, 40 um, 30 um, 20 um, 10 um, 5 um, 1 um or 0.5 um. In some instances, the width of a locus is less than about 100 um, 90 um, 80 um, 70 um, 60 um, 50 um, 40 um, 30 um, 20 um or 10 um. In some instances, the width of a locus is at least 10, 25, 50, 75, 100, 200, 300, 400, 500, 600, 700, 800, 900, 1000, or more than 1000 nm. In some instances, the width of a locus is in a range of about 10 nm to about 1000 nm, about 25 nm to about 900 nm, about 50 nm to about 800 nm, about 75 nm to about 700 nm, about 100 nm to about 600 nm, or about 200 nm to about 500. In some instances, the width of a locus is in a range of about 50 nm to about 1000 nm. In some instances, the distance between the center of two adjacent loci is from about 0.1 um to about 500 um, 0.5 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um. In some instances, the total width of a locus is about 5 um, 10 um, 20 um, 30 um, 40 um, 50 um, 60 um, 70 um, 80 um, 90 um, or 100 um. In some instances, the total width of a locus is about 1 um to 100 um, 30 um to 100 um, or 50 um to 70 um. In some instances, the distance between the center of two adjacent loci is from about 0.5 um to about 2 um, 0.5 um to about 2 um, from about 0.75 um to about 2 um, from about 1 um to about 2 um, from about 0.2 um to about 1 um, from about 0.5 um to about 1.5 um, from about 0.5 um to about 0.8 um, or from about 0.5 um to about 1 um, for example, about 1 um. In some instances, the total width of a locus is about 50 nm, 0.1 um, 0.2 um, 0.3 um, 0.4 um, 0.5 um, 0.6 um, 0.7 um, 0.8 um, 0.9 um, 1 um, 1.1 um, 1.2 um, 1.3 um, 1.4 um, or 1.5 um. In some instances, the total width of a locus is about 0.5 um to 2 um, 0.75 um to 1 um, or 0.9 um to 2 um.

In some instances, each locus supports the synthesis of a population of polynucleotides having a different sequence than a population of polynucleotides grown on another locus. Provided herein are surfaces which comprise at least 10, 100, 256, 500, 1000, 2000, 3000, 4000, 5000, 6000, 7000, 8000, 9000, 10000, 11000, 12000, 13000, 14000, 15000, 20000, 30000, 40000, 50000 or more clusters. Provided herein are surfaces which comprise more than 2,000; 5,000; 10,000; 20,000; 30,000; 50,000; 100,000; 200,000; 300,000; 400,000; 500,000; 600,000; 700,000; 800,000; 900,000; 1,000,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150, 200, 500 or more loci. In some cases, each cluster includes 50 to 500, 50 to 200, 50 to 150, or 100 to 150 loci. In some cases, each cluster includes 100 to 150 loci. In some instances, each cluster includes 109, 121, 130 or 137 loci.

Provided herein are loci having a width at the longest segment of 5 to 100 um. In some cases, the loci have a width at the longest segment of about 30, 35, 40, 45, 50, 55 or 60 um. In some cases, the loci are channels having multiple segments, wherein each segment has a center to center distance apart of 5 to 50 um. In some cases, the center to center distance apart for each segment is about 5, 10, 15, 20 or 25 um.

In some instances, the number of distinct polynucleotides synthesized on the surface of a structure described herein is dependent on the number of distinct loci available in the substrate. In some instances, the density of loci within a cluster of a substrate is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm², 10⁴ loci per mm², 10⁵ loci per mm², 10⁶ loci per mm², or more. In some cases, a substrate comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some cases, a substrate comprises from about 10⁴ loci per mm² to about 10⁵ mm². In some cases, a substrate comprises from about 10⁵ loci per mm² to about 10⁷ mm². In some cases, a substrate comprises at least 10⁵ loci per mm². In some cases, a substrate comprises at least 10⁶ loci per mm². In some cases, a substrate comprises at least 10⁷ loci per mm². In some cases, a substrate comprises from about 10⁴ loci per mm² to about 10⁵ mm². In some instances, the density of loci within a cluster of a substrate is at least or about 1 locus per um², 10 loci per um², 25 loci per um², 50 loci per um², 65 loci per um², 75 loci per um², 100 loci per um², 130 loci per um², 150 loci per um², 175 loci per um², 200 loci per um², 300 loci per um² 400 loci per um², 500 loci per um², 1,000 loci per um² or more. In some cases, a substrate comprises from about 10 loci per um² to about 500 um², from about 25 loci per um² to about 400 um², from about 50 loci per um² to about 500 um², from about 100 loci per um² to about 500 um², from about 150 loci per um² to about 500 um², from about 10 loci per um² to about 250 um², from about 50 loci per um² to about 250 um², from about 10 loci per um² to about 200 um², or from about 50 loci per um² to about 200 um².

In some instances, the distance between the centers of two adjacent loci within a cluster is from about 10 um to about 500 um, from about 10 um to about 200 um, or from about 10 um to about 100 um. In some cases, the distance between two centers of adjacent loci is greater than about 10 um, 20 um, 30 um, 40 um, 50 um, 60 um, 70 um, 80 um, 90 um or 100 um. In some cases, the distance between the centers of two adjacent loci is less than about 200 um, 150 um, 100 um, 80 um, 70 um, 60 um, 50 um, 40 um, 30 um, 20 um or 10 um. In some cases, the distance between the centers of two adjacent loci is less than about 10000 nm, 8000 nm, 6000 nm, 4000 nm, 2000 nm 1000 nm, 800 nm, 600 nm, 400 nm, 200 nm, 150 nm, 100 nm, 80 um, 70 nm, 60 nm, 50 nm, 40 nm, 30 nm, 20 nm or 10 nm. In some instances, each square meter of a structure described herein allows for at least 10⁷, 10⁸, 10⁹, 10¹⁰, 10¹¹ loci, where each locus supports one polynucleotide. In some instances, 10⁹ polynucleotides are supported on less than about 6, 5, 4, 3, 2 or 1 m² of a structure described herein.

In some instances, a structure described herein provides support for the synthesis of more than 2,000; 5,000; 10,000; 20,000; 30,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the structure provides support for the synthesis of more than 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the structure provides a surface environment for the growth of polynucleotides having at least 50, 60, 70, 75, 80, 85, 90, 95, 100, 110, 120, 130, 140, 150, 160, 175, 200, 225, 250, 275, 300, 325, 350, 375, 400, 425, 450, 475, 500 bases or more. In some arrangements, structures for polynucleotide synthesis described herein comprise sites for polynucleotide synthesis in a uniform arrangement.

In some instances, polynucleotides are synthesized on distinct loci of a structure, wherein each locus supports the synthesis of a population of polynucleotides. In some cases, each locus supports the synthesis of a population of polynucleotides having a different sequence than a population of polynucleotides grown on another locus. In some instances, the loci of a structure are located within a plurality of clusters. In some instances, a structure comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a structure comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some cases, each cluster includes 1, 2, 3, 4, 5, 6, 7, 8, 9, 10, 20, 30, 40, 50, 60, 70, 80, 90, 100, 120, 130, 150 or more loci. In some instances, each cluster includes 50 to 500, 100 to 150, or 100 to 200 loci. In some instances, each cluster includes 109, 121, 130 or 137 loci. In some instances, each cluster includes 5, 6, 7, 8, 9, 10, 11 or 12 loci. In some instances, polynucleotides from distinct loci within one cluster have sequences that, when assembled, encode for a contiguous longer polynucleotide of a predetermined sequence.

### Structure Size

In some instances, a structure described herein is about the size of a plate (e.g., chip), for example between about 40 and 120 mm by between about 25 and 100 mm. In some instances, a structure described herein has a diameter less than or equal to about 1000 mm, 500 mm, 450 mm, 400 mm, 300 mm, 250 nm, 200 mm, 150 mm, 100 mm or 50 mm. In some instances, the diameter of a substrate is between about 25 mm and 1000 mm, between about 25 mm and about 800 mm, between about 25 mm and about 600 mm, between about 25 mm and about 500 mm, between about 25 mm and about 400 mm, between about 25 mm and about 300 mm, or between about 25 mm and about 200. Non-limiting examples of substrate size include about 300 mm, 200 mm, 150 mm, 130 mm, 100 mm, 84 mm, 76 mm, 54 mm, 51 mm and 25 mm. In some instances, a substrate has a planar surface area of at least 100 mm²; 200 mm²; 500 mm²; 1,000 mm²; 2,000 mm²; 4,500 mm²; 5,000 mm²; 10,000 mm²; 12,000 mm²; 15,000 mm²; 20,000 mm²; 30,000 mm²; 40,000 mm²; 50,000 mm² or more. In some instances, the thickness is between about 50 mm and about 2000 mm, between about 50 mm and about 1000 mm, between about 100 mm and about 1000 mm, between about 200 mm and about 1000 mm, or between about 250 mm and about 1000 mm. Non-limiting examples thickness include 275 mm, 375 mm, 525 mm, 625 mm, 675 mm, 725 mm, 775 mm and 925 mm. In some instances, the thickness is at least or about 0.5 mm, 1.0 mm, 1.5 mm, 2.0 mm, 2.5 mm, 3.0 mm, 3.5 mm, 4.0 mm, or more than 4.0 mm. In some cases, the thickness of varies with diameter and depends on the composition of the substrate. For example, a structure comprising materials other than silicon may have a different thickness than a silicon structure of the same diameter. Structure thickness may be determined by the mechanical strength of the material used and the structure must be thick enough to support its own weight without cracking during handling. In some instances, a structure is more than about 1, 2, 3, 4, 5, 10, 15, 30, 40, 50 feet in any one dimension.

### Materials

Provided herein are devices comprising a surface, wherein the surface is modified to support polynucleotide synthesis at predetermined locations and with a resulting low error rate, a low dropout rate, a high yield, and a high oligo representation. In some instances, surfaces of devices for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the devices. Devices described herein may comprise a flexible material. Exemplary flexible materials include, without limitation, modified nylon, unmodified nylon, nitrocellulose, and polypropylene. Devices described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Devices disclosed herein may be fabricated from a material comprising silicon, polystyrene, agarose, dextran, cellulosic polymers, polyacrylamides, polydimethylsiloxane (PDMS), glass, or any combination thereof. In some cases, devices disclosed herein are manufactured with a combination of materials listed herein or any other suitable material known in the art.

Devices described herein may comprise material having a range of tensile strength. Exemplary materials having a range of tensile strengths include, but are not limited to, nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. Exemplary materials having a range of Young's modulus stiffness include, but are not limited to, nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load. In some instances, a solid support described herein has a surface with a flexibility of at least nylon.

In some cases, devices disclosed herein comprise a silicon dioxide base and a surface layer of silicon oxide. Alternatively, the devices may have a base of silicon oxide. Surface of the devices provided here may be textured, resulting in an increase overall surface area for polynucleotide synthesis. Devices disclosed herein in some instances comprise at least 5%, 10%, 25%, 50%, 80%, 90%, 95%, or 99% silicon. Devices disclosed herein in some instances are fabricated from silicon on insulator (SOI) wafer.

The structure may be fabricated from a variety of materials, suitable for the methods and compositions of the invention described herein. In instances, the materials from which the substrates/solid supports of the comprising the invention are fabricated exhibit a low level of polynucleotide binding. In some situations, material that are transparent to visible and/or UV light can be employed. Materials that are sufficiently conductive, e.g. those that can form uniform electric fields across all or a portion of the substrates/solids support described herein, can be utilized. In some instances, such materials may be connected to an electric ground. In some cases, the substrate or solid support can be heat conductive or insulated. The materials can be chemical resistant and heat resistant to support chemical or biochemical reactions such as a series of polynucleotide synthesis reactions. For flexible materials, materials of interest can include: nylon, both modified and unmodified, nitrocellulose, polypropylene, and the like.

For rigid materials, specific materials of interest include: glass; fuse silica; silicon, plastics (for example polytetrafluoroethylene, polypropylene, polystyrene, polycarbonate, and blends thereof, and the like); metals (for example, gold, platinum, and the like). The structure can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

In some instances, a substrate disclosed herein comprises a computer readable material. Computer readable materials include, without limitation, magnetic media, reel-to-reel tape, cartridge tape, cassette tape, flexible disk, paper media, film, microfiche, continuous tape (e.g., a belt) and any media suitable for storing electronic instructions. In some cases, the substrate comprises magnetic reel-to-reel tape or a magnetic belt. In some instances, the substrate comprises a flexible printed circuit board.

Structures described herein may be transparent to visible and/or UV light. In some instances, structures described herein are sufficiently conductive to form uniform electric fields across all or a portion of a structure. In some instances, structures described herein are heat conductive or insulated. In some instances, the structures are chemical resistant and heat resistant to support a chemical reaction such as a polynucleotide synthesis reaction. In some instances, the substrate is magnetic. In some instances, the structures comprise a metal or a metal alloy.

Structures for polynucleotide synthesis may be over 1, 2, 5, 10, 30, 50 or more feet long in any dimension. In the case of a flexible structure, the flexible structure is optionally stored in a wound state, e.g., in a reel. In the case of a large rigid structure, e.g., greater than 1 foot in length, the rigid structure can be stored vertically or horizontally.

### Surface Preparation

Provided herein are methods to support the immobilization of a biomolecule on a substrate, where a surface of a structure described herein comprises a material and/or is coated with a material that facilitates a coupling reaction with the biomolecule for attachment. To prepare a structure for biomolecule immobilization, surface modifications may be employed that chemically and/or physically alter the substrate surface by an additive or subtractive process to change one or more chemical and/or physical properties of a substrate surface or a selected site or region of the surface. For example, surface modification involves (1) changing the wetting properties of a surface, (2) functionalizing a surface, i.e. providing, modifying or substituting surface functional groups, (3) defunctionalizing a surface, i.e. removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface. In some instances, the surface of a structure is selectively functionalized to produce two or more distinct areas on a structure, wherein at least one area has a different surface or chemical property that another area of the same structure. Such properties include, without limitation, surface energy, chemical termination, surface concentration of a chemical moiety, and the like.

In some instances, a surface of a structure disclosed herein is modified to comprise one or more actively functionalized surfaces configured to bind to both the surface of the substrate and a biomolecule, thereby supporting a coupling reaction to the surface. In some instances, the surface is also functionalized with a passive material that does not efficiently bind the biomolecule, thereby preventing biomolecule attachment at sites where the passive functionalization agent is bound. In some cases, the surface comprises an active layer only defining distinct loci for biomolecule support.

In some instances, the surface is contacted with a mixture of functionalization groups which are in any different ratio. In some instances, a mixture comprises at least 2, 3, 4, 5 or more different types of functionalization agents. In some cases, the ratio of the at least two types of surface functionalization agents in a mixture is about 1:1, 1:2, 1:5, 1:10, 2:10, 3:10, 4:10, 5:10, 6:10, 7:10, 8:10, 9:10, or any other ratio to achieve a desired surface representation of two groups. In some instances, desired surface tensions, wettabilities, water contact angles, and/or contact angles for other suitable solvents are achieved by providing a substrate surface with a suitable ratio of functionalization agents. In some cases, the agents in a mixture are chosen from suitable reactive and inert moieties, thus diluting the surface density of reactive groups to a desired level for downstream reactions. In some instances, the mixture of functionalization reagents comprises one or more reagents that bind to a biomolecule and one or more reagents that do not bind to a biomolecule. Therefore, modulation of the reagents allows for the control of the amount of biomolecule binding that occurs at a distinct area of functionalization.

In some instances, a method for substrate functionalization comprises deposition of a silane molecule onto a surface of a substrate. The silane molecule may be deposited on a high energy surface of the substrate. In some instances the high surface energy region includes a passive functionalization reagent. Methods described herein provide for a silane group to bind the surface, while the rest of the molecule provides a distance from the surface and a free hydroxyl group at the end to which a biomolecule attaches. In some instances, the silane is an organofunctional alkoxysilane molecule. Non-limiting examples of organofunctional alkoxysilane molecules include dimethylchloro-octodecyl-silane, methyldichloro-octodecyl-silane, trichloro-octodecyl-silane, and trimethyl-octodecyl-silane, triethyl-octodecyl-silane. In some instances, the silane is an amino silane. Examples of amino silanes include, without limitation, 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide. In some instances, the silane comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane, N-(3-triethoxysilylpropyl)-4-hydroxybutyramide, or any combination thereof. In some instances, an active functionalization agent comprises 11-acetoxyundecyltriethoxysilane. In some instances, an active functionalization agent comprises n-decyltriethoxysilane. In some cases, an active functionalization agent comprises glycidyloxypropyltriethoxysilane (GOPS). In some instances, the silane is a fluorosilane. In some instances, the silane is a hydrocarbon silane. In some cases, the silane is 3-iodo-propyltrimethoxysilane. In some cases, the silane is octylchlorosilane.

In some instances, silanization is performed on a surface through self-assembly with organofunctional alkoxysilane molecules. The organofunctional alkoxysilanes are classified according to their organic functions. Non-limiting examples of siloxane functionalizing reagents include hydroxyalkyl siloxanes (silylate surface, functionalizing with diborane and oxidizing the alcohol by hydrogen peroxide), diol (dihydroxyalkyl) siloxanes (silylate surface, and hydrolyzing to diol), aminoalkyl siloxanes (amines require no intermediate functionalizing step), glycidoxysilanes (3-glycidoxypropyl-dimethyl-ethoxysilane, glycidoxy-trimethoxysilane), mercaptosilanes (3-mercaptopropyl-trimethoxysilane, 3-4 epoxycyclohexyl-ethyltrimethoxysilane or 3-mercaptopropyl-methyl-dimethoxysilane), bicyclohepthenyl-trichlorosilane, butyl-aldehydr-trimethoxysilane, or dimeric secondary aminoalkyl siloxanes. Exemplary hydroxyalkyl siloxanes include allyl trichlorochlorosilane turning into 3-hydroxypropyl, or 7-oct-1-enyl trichlorochlorosilane turning into 8-hydroxyoctyl. The diol (dihydroxyalkyl) siloxanes include glycidyl trimethoxysilane-derived (2,3-dihydroxypropyloxy)propyl (GOPS). The aminoalkyl siloxanes include 3-aminopropyl trimethoxysilane turning into 3-aminopropyl (3-aminopropyl-triethoxysilane, 3-aminopropyl-diethoxy-methylsilane, 3-aminopropyl-dimethyl-ethoxysilane, or 3-aminopropyl-trimethoxysilane). In some cases, the dimeric secondary aminoalkyl siloxanes is bis (3-trimethoxysilylpropyl) amine turning into bis(silyloxylpropyl)amine.

Active functionalization areas may comprise one or more different species of silanes, for example, 1, 2, 3, 4, 5, 6, 7, 8, 9, 10 or more silanes. In some cases, one of the one or more silanes is present in the functionalization composition in an amount greater than another silane. For example, a mixed silane solution having two silanes comprises a 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 75:25, 70:30, 65:35, 60:40, 55:45 ratio of one silane to another silane. In some instances, an active functionalization agent comprises 11-acetoxyundecyltriethoxysilane and n-decyltriethoxysilane. In some instances, an active functionalization agent comprises 11-acetoxyundecyltriethoxysilane and n-decyltriethoxysilane in a ratio from about 20:80 to about 1:99, or about 10:90 to about 2:98, or about 5:95.

In some instances, functionalization comprises deposition of a functionalization agent to a structure by any deposition technique, including, but not limiting to, chemical vapor deposition (CVD), atomic layer deposition (ALD), plasma enhanced CVD (PECVD), plasma enhanced ALD (PEALD), metal organic CVD (MOCVD), hot wire CVD (HWCVD), initiated CVD (iCVD), modified CVD (MCVD), vapor axial deposition (VAD), outside vapor deposition (OVD), physical vapor deposition (e.g., sputter deposition, evaporative deposition), and molecular layer deposition (MLD).

Any step or component in the following functionalization process be omitted or changed in accordance with properties desired of the final functionalized substrate. In some cases, additional components and/or process steps are added to the process workflows embodied herein. In some instances, a substrate is first cleaned, for example, using a piranha solution. An example of a cleaning process includes soaking a substrate in a piranha solution (e.g., 90% H₂SO₄, 10% H₂O₂) at an elevated temperature (e.g., 120° C.) and washing (e.g., water) and drying the substrate (e.g., nitrogen gas). The process optionally includes a post piranha treatment comprising soaking the piranha treated substrate in a basic solution (e.g., NH₄OH) followed by an aqueous wash (e.g., water). In some instances, a surface of a structure is plasma cleaned, optionally following the piranha soak and optional post piranha treatment. An example of a plasma cleaning process comprises an oxygen plasma etch. In some instances, the surface is deposited with an active functionalization agent following by vaporization. In some instances, the substrate is actively functionalized prior to cleaning, for example, by piranha treatment and/or plasma cleaning.

The process for surface functionalization optionally comprises a resist coat and a resist strip. In some instances, following active surface functionalization, the substrate is spin coated with a resist, for example, SPR™ 3612 positive photoresist. The process for surface functionalization, in various instances, comprises lithography with patterned functionalization. In some instances, photolithography is performed following resist coating. In some instances, after lithography, the surface is visually inspected for lithography defects. The process for surface functionalization, in some instances, comprises a cleaning step, whereby residues of the substrate are removed, for example, by plasma cleaning or etching. In some instances, the plasma cleaning step is performed at some step after the lithography step.

In some instances, a surface coated with a resist is treated to remove the resist, for example, after functionalization and/or after lithography. In some cases, the resist is removed with a solvent, for example, with a stripping solution comprising N-methyl-2-pyrrolidone. In some cases, resist stripping comprises sonication or ultrasonication. In some instances, a resist is coated and stripped, followed by active functionalization of the exposed areas to create a desired differential functionalization pattern.

In some instances, the methods and compositions described herein relate to the application of photoresist for the generation of modified surface properties in selective areas, wherein the application of the photoresist relies on the fluidic properties of the surface defining the spatial distribution of the photoresist. Without being bound by theory, surface tension effects related to the applied fluid may define the flow of the photoresist. For example, surface tension and/or capillary action effects may facilitate drawing of the photoresist into small structures in a controlled fashion before the resist solvents evaporate. In some instances, resist contact points are pinned by sharp edges, thereby controlling the advance of the fluid. The underlying structures may be designed based on the desired flow patterns that are used to apply photoresist during the manufacturing and functionalization processes. A solid organic layer left behind after solvents evaporate may be used to pursue the subsequent steps of the manufacturing process. Structures may be designed to control the flow of fluids by facilitating or inhibiting wicking effects into neighboring fluidic paths. For example, a structure is designed to avoid overlap between top and bottom edges, which facilitates the keeping of the fluid in top structures allowing for a particular disposition of the resist. In an alternative example, the top and bottom edges overlap, leading to the wicking of the applied fluid into bottom structures. Appropriate designs may be selected accordingly, depending on the desired application of the resist.

In some instances, a structure described herein has a surface that comprises a material having thickness of at least or at least 0.1 nm, 0.5 nm, 1 nm, 2 nm, 5 nm, 10 nm or 25 nm that comprises a reactive group capable of binding nucleosides. Exemplary include, without limitation, glass and silicon, such as silicon dioxide and silicon nitride. In some cases, exemplary surfaces include nylon and PMMA.

In some instances, electromagnetic radiation in the form of UV light is used for surface patterning. In some instances, a lamp is used for surface patterning, and a mask mediates exposure locations of the UV light to the surface. In some instances, a laser is used for surface patterning, and a shutter opened/closed state controls exposure of the UV light to the surface. The laser arrangement may be used in combination with a flexible structure that is capable of moving. In such an arrangement, the coordination of laser exposure and flexible structure movement is used to create patterns of one or more agents having differing nucleoside coupling capabilities.

Described herein are surfaces for polynucleotide synthesis that are reusable. After synthesis and/or cleavage of polynucleotides, a surface may be bathed, washed, cleaned, baked, etched, or otherwise functionally restored to a condition suitable for subsequent polynucleotide synthesis. The number of times a surface is reused and the methods for recycling/preparing the surface for reuse vary depending on subsequent applications. Surfaces prepared for reuse are in some instances reused at least 1, 2, 3, 5, 10, 20, 50, 100, 1,000 or more times. In some instances, the remaining “life” or number of times a surface is suitable for reuse is measured or predicted.

### Material Deposition Systems

In some cases, the synthesized polynucleotides are stored on the substrate, for example a solid support. Nucleic acid reagents may be deposited on the substrate surface in a non-continuous, or drop-on-demand method. Examples of such methods include the electromechanical transfer method, electric thermal transfer method, and electrostatic attraction method. In the electromechanical transfer method, piezoelectric elements deformed by electrical pulses cause the droplets to be ejected. In the electric thermal transfer method, bubbles are generated in a chamber of the device, and the expansive force of the bubbles causes the droplets to be ejected. In the electrostatic attraction method, electrostatic force of attraction is used to eject the droplets onto the substrate. In some cases, the drop frequency is from about 5 KHz to about 500 KHz; from about 5 KHz to about 100 KHz; from about 10 KHz to about 500 KHz; from about 10 KHz to about 100 KHz; or from about 50 KHz to about 500 KHz. In some cases, the frequency is less than about 500 KHz, 200 KHz, 100 KHz, or 50 KHz.

The size of the droplets dispensed correlates to the resolution of the device. In some instances, the devices deposit droplets of reagents at sizes from about 0.01 pl to about 20 pl, from about 0.01 pl to about 10 pl, from about 0.01 pl to about 1 pl, from about 0.01 pl to about 0.5 pl, from about 0.01 pl to about 0.01 pl, or from about 0.05 pl to about 1 pl. In some instances, the droplet size is less than about 1 pl, 0.5 pl, 0.2 pl, 0.1 pl, or 0.05 pl.

In some arrangements, the configuration of a polynucleotide synthesis system allows for a continuous polynucleotide synthesis process that exploits the flexibility of a substrate for traveling in a reel-to-reel type process. This synthesis process operates in a continuous production line manner with the substrate travelling through various stages of polynucleotide synthesis using one or more reels to rotate the position of the substrate. In an exemplary instance, a polynucleotide synthesis reaction comprises rolling a substrate: through a solvent bath, beneath a deposition device for phosphoramidite deposition, through a bath of oxidizing agent, through an acetonitrile wash bath, and through a deblock bath. Optionally, the tape is also traversed through a capping bath. A reel-to-reel type process allows for the finished product of a substrate comprising synthesized polynucleotides to be easily gathered on a take-up reel, where it can be transported for further processing or storage.

In some arrangements, polynucleotide synthesis proceeds in a continuous process as a continuous flexible tape is conveyed along a conveyor belt system. Similar to the reel-to-reel type process, polynucleotide synthesis on a continuous tape operates in a production line manner, with the substrate travelling through various stages of polynucleotide synthesis during conveyance. However, in a conveyor belt process, the continuous tape revisits a polynucleotide synthesis step without rolling and unrolling of the tape, as in a reel-to-reel process. In some arrangements, polynucleotide synthesis steps are partitioned into zones and a continuous tape is conveyed through each zone one or more times in a cycle. For example, a polynucleotide synthesis reaction may comprise (1) conveying a substrate through a solvent bath, beneath a deposition device for phosphoramidite deposition, through a bath of oxidizing agent, through an acetonitrile wash bath, and through a block bath in a cycle; and then (2) repeating the cycles to achieve synthesized polynucleotides of a predetermined length. After polynucleotide synthesis, the flexible substrate is removed from the conveyor belt system and, optionally, rolled for storage. Rolling may be around a reel, for storage. In some instances, a flexible substrate comprising thermoplastic material is coated with nucleoside coupling reagent. The coating is patterned into loci such that each locus has diameter of about 10 um, with a center-to-center distance between two adjacent loci of about 21 um. In this instance, the locus size is sufficient to accommodate a sessile drop volume of 0.2 pl during a polynucleotide synthesis deposition step. In some cases, the locus density is about 2.2 billion loci per m² (1 locus/441×10⁻¹² m²). In some cases, a 4.5 m² substrate comprise about 10 billion loci, each with a 10 um diameter.

In some arrangements, a device for application of one or more reagents to a substrate during a synthesis reaction is configured to deposit reagents and/or nucleoside monomers for nucleoside phosphoramidite based synthesis. Reagents for polynucleotide synthesis include reagents for polynucleotide extension and wash buffers. As non-limiting examples, the device deposits cleaning reagents, coupling reagents, capping reagents, oxidizers, de-blocking agents, acetonitrile, gases such as nitrogen gas, and any combination thereof. In addition, the device optionally deposits reagents for the preparation and/or maintenance of substrate integrity. In some instances, the polynucleotide synthesizer deposits a drop having a diameter less than about 200 um, 100 um, or 50 um in a volume less than about 1000, 500, 100, 50, or 20 pl. In some cases, the polynucleotide synthesizer deposits between about 1 and 10000, 1 and 5000, 100 and 5000, or 1000 and 5000 droplets per second.

Described herein are devices, methods, systems and compositions where reagents for polynucleotide synthesis are recycled or reused. Recycling of reagents may comprise collection, storage, and usage of unused reagents, or purification/transformation of used reagents. For example, a reagent bath is recycled and used for a polynucleotide synthesis step on the same or a different surface. Reagents described herein may be recycled 1, 2, 3, 4, 5, 6, 7, 8, 9, 10 or more times. Alternatively or in combination, a reagent solution comprising a reaction byproduct is filtered to remove the byproduct, and the reagent solution is used for additional polynucleotide synthesis reactions.

Many integrated or non-integrated elements are often used with polynucleotide synthesis systems. In some instances, a polynucleotide synthesis system comprises one or more elements useful for downstream processing of synthesized polynucleotides. As an example, the system comprises a temperature control element such as a thermal cycling device. In some instances, the temperature control element is used with a plurality of resolved reactors to perform nucleic acid assembly such as PCA and/or nucleic acid amplification such as PCR.

### De Novo Polynucleotide Synthesis

Provided herein are systems and methods for synthesis of a high density of polynucleotides on a substrate in a short amount of time. In some instances, the substrate is a flexible substrate. In some instances, at least 10¹⁰, 10¹¹, 10¹², 10¹³, 10¹⁴, or 10¹⁵ bases are synthesized in one day. In some instances, at least 10×10⁸, 10×10⁹, 10×10¹⁰, 10×10¹¹, or 10×10¹² polynucleotides are synthesized in one day. In some cases, each polynucleotide synthesized comprises at least 20, 50, 100, 200, 300, 400 or 500 nucleobases. In some cases, these bases are synthesized with a total average error rate of less than about 1 in 100; 200; 300; 400; 500; 1000; 2000; 5000; 10000; 15000; 20000 bases. In some instances, these error rates are for at least 50%, 60%, 70%, 80%, 90%, 95%, 98%, 99%, 99.5%, or more of the polynucleotides synthesized. In some instances, these at least 90%, 95%, 98%, 99%, 99.5%, or more of the polynucleotides synthesized do not differ from a predetermined sequence for which they encode. In some instances, the error rate for synthesized polynucleotides on a substrate using the methods and systems described herein is less than about 1 in 200. In some instances, the error rate for synthesized polynucleotides on a substrate using the methods and systems described herein is less than about 1 in 1,000. In some instances, the error rate for synthesized polynucleotides on a substrate using the methods and systems described herein is less than about 1 in 2,000. In some instances, the error rate for synthesized polynucleotides on a substrate using the methods and systems described herein is less than about 1 in 3,000. In some instances, the error rate for synthesized polynucleotides on a substrate using the methods and systems described herein is less than about 1 in 5,000. Individual types of error rates include mismatches, deletions, insertions, and/or substitutions for the polynucleotides synthesized on the substrate. The term “error rate” refers to a comparison of the collective amount of synthesized polynucleotide to an aggregate of predetermined polynucleotide sequences. In some instances, synthesized polynucleotides disclosed herein comprise a tether of 12 to 25 bases. In some instances, the tether comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more bases.

Described herein are methods, systems, devices, and compositions wherein chemical reactions used in polynucleotide synthesis are controlled using electrochemistry. Electrochemical reactions in some instances are controlled by any source of energy, such as light, heat, radiation, or electricity. For example, electrodes are used to control chemical reactions as all or a portion of discrete loci on a surface. Electrodes in some instances are charged by applying an electrical potential to the electrode to control one or more chemical steps in polynucleotide synthesis. In some instances, these electrodes are addressable. Any number of the chemical steps described herein is in some instances controlled with one or more electrodes. Electrochemical reactions may comprise oxidations, reductions, acid/base chemistry, or other reaction that is controlled by an electrode. In some instances, electrodes generate electrons or protons that are used as reagents for chemical transformations. Electrodes in some instances directly generate a reagent such as an acid. In some instances, an acid is a proton. Electrodes in some instances directly generate a reagent such as a base. Acids or bases are often used to cleave protecting groups, or influence the kinetics of various polynucleotide synthesis reactions, for example by adjusting the pH of a reaction solution. Electrochemically controlled polynucleotide synthesis reactions in some instances comprise redox-active metals or other redox-active organic materials. In some instances, metal or organic catalysts are employed with these electrochemical reactions. In some instances, acids are generated from oxidation of quinones.

Control of chemical reactions with is not limited to the electrochemical generation of reagents; chemical reactivity may be influenced indirectly through biophysical changes to substrates or reagents through electric fields (or gradients) which are generated by electrodes. In some instances, substrates include but are not limited to nucleic acids. In some instances, electrical fields which repel or attract specific reagents or substrates towards or away from an electrode or surface are generated. Such fields in some instances are generated by application of an electrical potential to one or more electrodes. For example, negatively charged nucleic acids are repelled from negatively charged electrode surfaces. Such repulsions or attractions of polynucleotides or other reagents caused by local electric fields in some instances provides for movement of polynucleotides or other reagents in or out of region of the synthesis device or structure. In some instances, electrodes generate electric fields which repel polynucleotides away from a synthesis surface, structure, or device. In some instances, electrodes generate electric fields which attract polynucleotides towards a synthesis surface, structure, or device. In some instances, protons are repelled from a positively charged surface to limit contact of protons with substrates or portions thereof. In some instances, repulsion or attractive forces are used to allow or block entry of reagents or substrates to specific areas of the synthesis surface. In some instances, nucleoside monomers are prevented from contacting a polynucleotide chain by application of an electric field in the vicinity of one or both components. Such arrangements allow gating of specific reagents, which may obviate the need for protecting groups when the concentration or rate of contact between reagents and/or substrates is controlled. In some instances, unprotected nucleoside monomers are used for polynucleotide synthesis. Alternatively, application of the field in the vicinity of one or both components promotes contact of nucleoside monomers with a polynucleotide chain. Additionally, application of electric fields to a substrate can alter the substrates reactivity or conformation. In an exemplary application, electric fields generated by electrodes are used to prevent polynucleotides at adjacent loci from interacting. In some instances, the substrate is a polynucleotide, optionally attached to a surface. Application of an electric field in some instances alters the three-dimensional structure of a polynucleotide. Such alterations comprise folding or unfolding of various structures, such as helices, hairpins, loops, or other 3-dimensional nucleic acid structure. Such alterations are useful for manipulating nucleic acids inside of wells, channels, or other structures. In some instances, electric fields are applied to a nucleic acid substrate to prevent secondary structures. In some instances, electric fields obviate the need for linkers or attachment to a solid support during polynucleotide synthesis.

A suitable method for polynucleotide synthesis on a substrate of this disclosure is a phosphoramidite method comprising the controlled addition of a phosphoramidite building block, i.e. nucleoside phosphoramidite, to a growing polynucleotide chain in a coupling step that forms a phosphite triester linkage between the phosphoramidite building block and a nucleoside bound to the substrate. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition and linkage of a nucleoside phosphoramidite in the coupling step, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. Protecting groups may comprise any chemical group that prevents extension of the polynucleotide chain. In some instances, the protecting group is cleaved (or removed) in the presence of an acid. In some instances, the protecting group is cleaved in the presence of a base. In some instances, the protecting group is removed with electromagnetic radiation such as light, heat, or other energy source. In some instances, the protecting group is removed through an oxidation or reduction reaction. In some instances, a protecting group comprises a triarylmethyl group. In some instances, a protecting group comprises an aryl ether. In some instances, a protecting comprises a disulfide. In some instances a protecting group comprises an acid-labile silane. In some instances, a protecting group comprises an acetal. In some instances, a protecting group comprises a ketal. In some instances, a protecting group comprises an enol ether. In some instances, a protecting group comprises a methoxybenzyl group. In some instances, a protecting group comprises an azide. In some instances, a protecting group is 4,4′-dimethoxytrityl (DMT). In some instances, a protecting group is a tert-butyl carbonate. In some instances, a protecting group is a tert-butyl ester. In some instances, a protecting group comprises a base-labile group.

Following coupling, phosphoramidite polynucleotide synthesis methods optionally comprise a capping step. In a capping step, the growing polynucleotide is treated with a capping agent. A capping step generally serves to block unreacted substrate-bound 5′-OH groups after coupling from further chain elongation, preventing the formation of polynucleotides with internal base deletions. Further, phosphoramidites activated with 1H-tetrazole often react, to a small extent, with the O6 position of guanosine. Without being bound by theory, upon oxidation with I₂/water, this side product, possibly via O6-N7 migration, undergoes depurination. The apurinic sites can end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

Following addition of a nucleoside phosphoramidite, and optionally after capping and one or more wash steps, a substrate described herein comprises a bound growing nucleic acid that may be oxidized. The oxidation step comprises oxidizing the phosphite triester into a tetracoordinated phosphate triester, a protected precursor of the naturally occurring phosphate diester intemucleoside linkage. In some instances, phosphite triesters are oxidized electrochemically. In some instances, oxidation of the growing polynucleotide is achieved by treatment with iodine and water, optionally in the presence of a weak base such as a pyridine, lutidine, or collidine. Oxidation is sometimes carried out under anhydrous conditions using tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including, but not limited to, 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

For a subsequent cycle of nucleoside incorporation to occur through coupling, a protected 5′ end (or 3′ end, if synthesis is conducted in a 5′ to 3′ direction) of the substrate bound growing polynucleotide is be removed so that the primary hydroxyl group can react with a next nucleoside phosphoramidite. In some instances, the protecting group is DMT and deblocking occurs with trichloroacetic acid in dichloromethane. In some instances, the protecting group is DMT and deblocking occurs with electrochemically generated protons. Conducting detritylation for an extended time or with stronger than recommended solutions of acids may lead to increased depurination of solid support-bound polynucleotide and thus reduces the yield of the desired full-length product. Methods and compositions described herein provide for controlled deblocking conditions limiting undesired depurination reactions. In some instances, the substrate bound polynucleotide is washed after deblocking. In some cases, efficient washing after deblocking contributes to synthesized polynucleotides having a low error rate.

Methods for the synthesis of polynucleotides on a substrate described herein may involve an iterating sequence of the following steps: application of a protected monomer to a surface of a substrate feature to link with either the surface, a linker or with a previously deprotected monomer; deprotection of the applied monomer so that it can react with a subsequently applied protected monomer; and application of another protected monomer for linking. One or more intermediate steps include oxidation and/or sulfurization. In some instances, one or more wash steps precede or follow one or all of the steps.

Methods for the synthesis of polynucleotides on a substrate described herein may comprise an oxidation step. For example, methods involve an iterating sequence of the following steps: application of a protected monomer to a surface of a substrate feature to link with either the surface, a linker or with a previously deprotected monomer; deprotection of the applied monomer so that it can react with a subsequently applied protected monomer; application of another protected monomer for linking, and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; and oxidation and/or sulfurization. In some instances, one or more wash steps precede or follow one or all of the steps.

Methods for the synthesis of polynucleotides on a substrate described herein may further comprise an iterating sequence of the following steps: application of a protected monomer to a surface of a substrate feature to link with either the surface, a linker or with a previously deprotected monomer; deprotection of the applied monomer so that it can react with a subsequently applied protected monomer; and oxidation and/or sulfurization. In some instances, one or more wash steps precede or follow one or all of the steps.

In some instances, polynucleotides are synthesized with photolabile protecting groups, where the hydroxyl groups generated on the surface are blocked by photolabile-protecting groups. When the surface is exposed to UV light, such as through a photolithographic mask, a pattern of free hydroxyl groups on the surface may be generated. These hydroxyl groups can react with photoprotected nucleoside phosphoramidites, according to phosphoramidite chemistry. A second photolithographic mask can be applied and the surface can be exposed to UV light to generate second pattern of hydroxyl groups, followed by coupling with 5′-photoprotected nucleoside phosphoramidite. Likewise, patterns can be generated and oligomer chains can be extended. Without being bound by theory, the lability of a photocleavable group depends on the wavelength and polarity of a solvent employed and the rate of photocleavage may be affected by the duration of exposure and the intensity of light. This method can leverage a number of factors such as accuracy in alignment of the masks, efficiency of removal of photo-protecting groups, and the yields of the phosphoramidite coupling step. Further, unintended leakage of light into neighboring sites can be minimized. The density of synthesized oligomer per spot can be monitored by adjusting loading of the leader nucleoside on the surface of synthesis.

The surface of a substrate described herein that provides support for polynucleotide synthesis may be chemically modified to allow for the synthesized polynucleotide chain to be cleaved from the surface. In some instances, the polynucleotide chain is cleaved at the same time as the polynucleotide is deprotected. In some cases, the polynucleotide chain is cleaved after the polynucleotide is deprotected. In an exemplary scheme, a trialkoxysilyl amine such as (CH₃CH₂O)₃Si—(CH₂)₂—NH₂ is reacted with surface SiOH groups of a substrate, followed by reaction with succinic anhydride with the amine to create an amide linkage and a free OH on which the nucleic acid chain growth is supported. Cleavage includes gas cleavage with ammonia or methylamine. In some instances cleavage includes linker cleavage with electrically generated reagents such as acids or bases. In some instances, once released from the surface, polynucleotides are assembled into larger nucleic acids that are sequenced and decoded to extract stored information.

The surfaces described herein can be reused after polynucleotide cleavage to support additional cycles of polynucleotide synthesis. For example, the linker can be reused without additional treatment/chemical modifications. In some instances, a linker is non-covalently bound to a substrate surface or a polynucleotide. In some embodiments, the linker remains attached to the polynucleotide after cleavage from the surface. Linkers in some embodiments comprise reversible covalent bonds such as esters, amides, ketals, beta substituted ketones, heterocycles, or other group that is capable of being reversibly cleaved. Such reversible cleavage reactions are in some instances controlled through the addition or removal of reagents, or by electrochemical processes controlled by electrodes. Optionally, chemical linkers or surface-bound chemical groups are regenerated after a number of cycles, to restore reactivity and remove unwanted side product formation on such linkers or surface-bound chemical groups.

### Assembly

Polynucleotides may be designed to collectively span a large region of a predetermined sequence that encodes for information. In some instances, larger polynucleotides are generated through ligation reactions to join the synthesized polynucleotides. One example of a ligation reaction is polymerase chain assembly (PCA). In some instances, at least of a portion of the polynucleotides are designed to include an appended region that is a substrate for universal primer binding. For PCA reactions, the presynthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some cases, after the PCA reaction is complete, an error correction step is conducted using mismatch repair detecting enzymes to remove mismatches in the sequence. Once larger fragments of a target sequence are generated, they can be amplified. For example, in some cases, a target sequence comprising 5′ and 3′ terminal adapter sequences is amplified in a polymerase chain reaction (PCR) which includes modified primers that hybridize to the adapter sequences. In some cases, the modified primers comprise one or more uracil bases. The use of modified primers allows for removal of the primers through enzymatic reactions centered on targeting the modified base and/or gaps left by enzymes which cleave the modified base pair from the fragment. What remains is a double-stranded amplification product that lacks remnants of adapter sequence. In this way, multiple amplification products can be generated in parallel with the same set of primers to generate different fragments of double-stranded DNA.

Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some cases, DNA mismatch-binding protein MutS (Thermus aquaticus) is used to remove failure products from a population of synthesized products. In some instances, error correction is performed using the enzyme Correctase. In some cases, error correction is performed using SURVEYOR endonuclease (Transgenomic), a mismatch-specific DNA endonuclease that scans for known and unknown mutations and polymorphisms for heteroduplex DNA.

### Sequencing

After extraction and/or amplification of polynucleotides from the surface of the structure, suitable sequencing technology may be employed to sequence the polynucleotides. In some cases, the DNA sequence is read on the substrate or within a feature of a structure. In some cases, the polynucleotides stored on the substrate are extracted is optionally assembled into longer nucleic acids and then sequenced.

Polynucleotides synthesized and stored on the structures described herein encode data that can be interpreted by reading the sequence of the synthesized polynucleotides and converting the sequence into binary code readable by a computer. In some cases the sequences require assembly, and the assembly step may need to be at the nucleic acid sequence stage or at the digital sequence stage.

Provided herein are detection systems comprising a device capable of sequencing stored polynucleotides, either directly on the structure and/or after removal from the main structure. In cases where the structure is a reel-to-reel tape of flexible material, the detection system comprises a device for holding and advancing the structure through a detection location and a detector disposed proximate the detection location for detecting a signal originated from a section of the tape when the section is at the detection location. In some instances, the signal is indicative of a presence of a polynucleotide. In some instances, the signal is indicative of a sequence of a polynucleotide (e.g., a fluorescent signal). In some instances, information encoded within polynucleotides on a continuous tape is read by a computer as the tape is conveyed continuously through a detector operably connected to the computer. In some instances, a detection system comprises a computer system comprising a polynucleotide sequencing device, a database for storage and retrieval of data relating to polynucleotide sequence, software for converting DNA code of a polynucleotide sequence to binary code, a computer for reading the binary code, or any combination thereof.

Provided herein are sequencing systems that can be integrated into the devices described herein. Various methods of sequencing are well known in the art, and comprise “base calling” wherein the identity of a base in the target polynucleotide is identified. In some instances, polynucleotides synthesized using the methods, devices, compositions, and systems described herein are sequenced after cleavage from the synthesis surface. In some instances, sequencing occurs during or simultaneously with polynucleotide synthesis, wherein base calling occurs immediately after or before extension of a nucleoside monomer into the growing polynucleotide chain. Methods for base calling include measurement of electrical currents generated by polymerase-catalyzed addition of bases to a template strand. In some instances, synthesis surfaces comprise enzymes, such as polymerases. In some instances, such enzymes are tethered to electrodes or to the synthesis surface.

### Computer Systems

In various aspects, any of the systems described herein are operably linked to a computer and are optionally automated through a computer either locally or remotely. In various instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. In some instances, the computer systems are programmed to interface between the user specified base sequence and the position of a material deposition device to deliver the correct reagents to specified regions of the substrate.

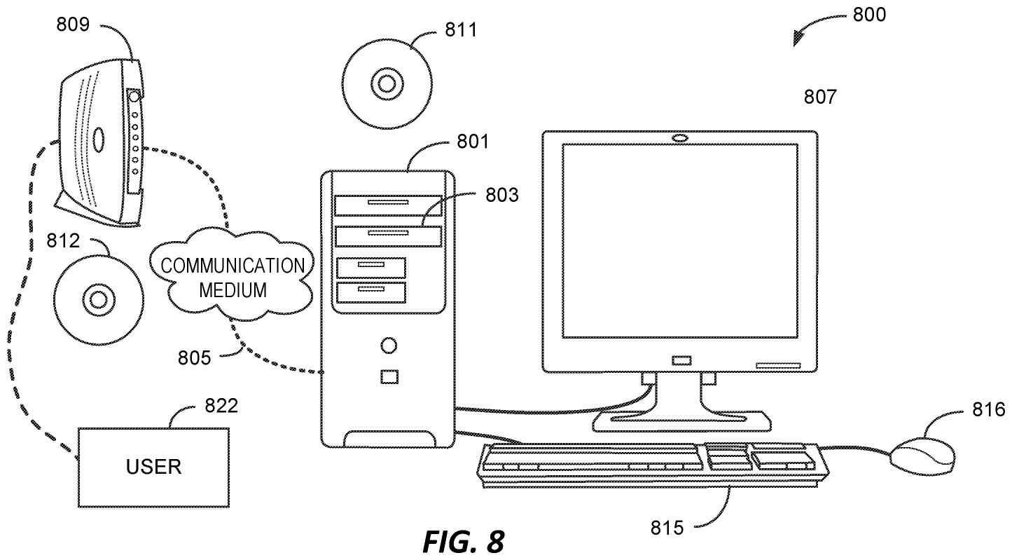

The computer system 800 illustrated in FIG. 8 may be understood as a logical apparatus that can read instructions from media 811 and/or a network port 805, which can optionally be connected to server 809 having fixed media 812. The system can include a CPU 801, disk drives 803, optional input devices such as keyboard 815 and/or mouse 816 and optional monitor 807. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 822.

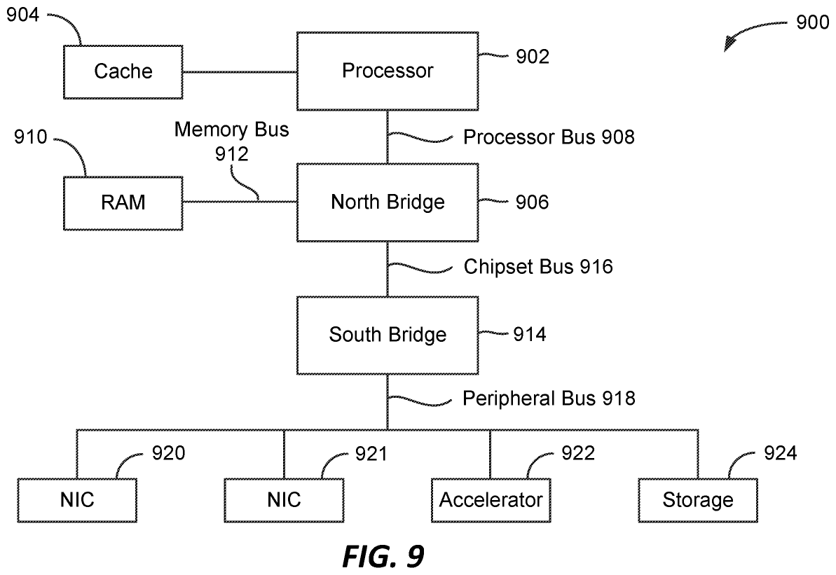

FIG. 9 is a block diagram illustrating a first example architecture of a computer system that can be used in connection with example instances of the present invention. As depicted in FIG. 5, the example computer system can include a processor 902 for processing instructions. Non-limiting examples of processors include: Intel Xeon™ processor, AMD Opteron™ processor, Samsung 32-bit RISC ARM 1176JZ(F)—S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 9, a high speed cache 904 can be connected to, or incorporated in, the processor 902 to provide a high speed memory for instructions or data that have been recently, or are frequently, used by processor 902. The processor 902 is connected to a north bridge 906 by a processor bus 908. The north bridge 906 is connected to random access memory (RAM) 910 by a memory bus 912 and manages access to the RAM 910 by the processor 902. The north bridge 906 is also connected to a south bridge 914 by a chipset bus 916. The south bridge 914 is, in turn, connected to a peripheral bus 918. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 918. In some alternative architectures, the functionality of the north bridge can be incorporated into the processor instead of using a separate north bridge chip.

In some instances, a system 900 can include an accelerator card 922 attached to the peripheral bus 918. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 924 and can be loaded into RAM 910 and/or cache 904 for use by the processor. The system 900 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention.

In this example, system 900 also includes network interface cards (NICs) 920 and 921 connected to the peripheral bus for providing network interfaces to external storage, such as Network Attached Storage (NAS) and other computer systems that can be used for distributed parallel processing.

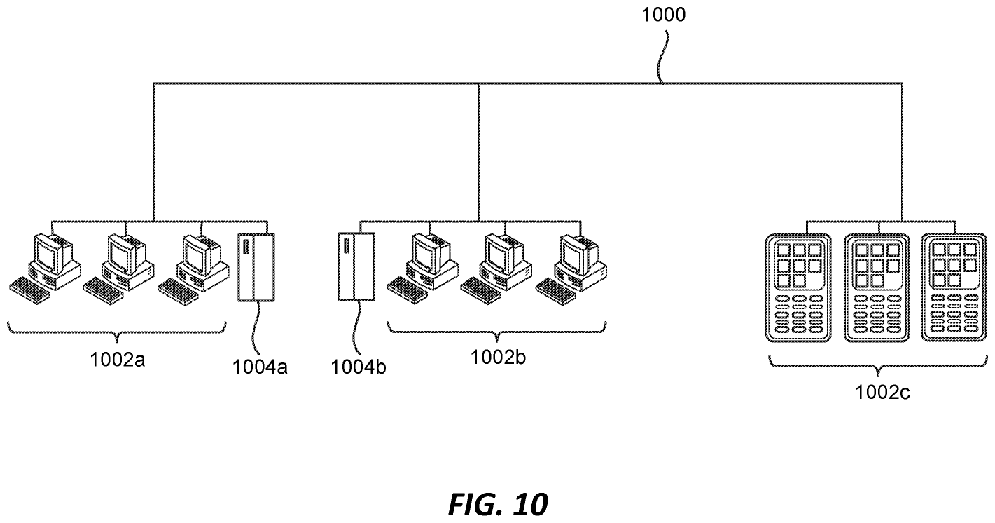

FIG. 10 is a diagram showing a network 1000 with a plurality of computer systems 1002a, and 1002b, a plurality of cell phones and personal data assistants 1002c, and Network Attached Storage (NAS) 1004a, and 1004b. In example embodiments, systems 1002a, 1002b, and 1002c can manage data storage and optimize data access for data stored in Network Attached Storage (NAS) 1004a and 1004b. A mathematical model can be used for the data and be evaluated using distributed parallel processing across computer systems 1002a, and 1002b, and cell phone and personal data assistant systems 1002c. Computer systems 1002a, and 1002b, and cell phone and personal data assistant systems 1002c can also provide parallel processing for adaptive data restructuring of the data stored in Network Attached Storage (NAS) 1004a and 1004b. FIG. 10 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

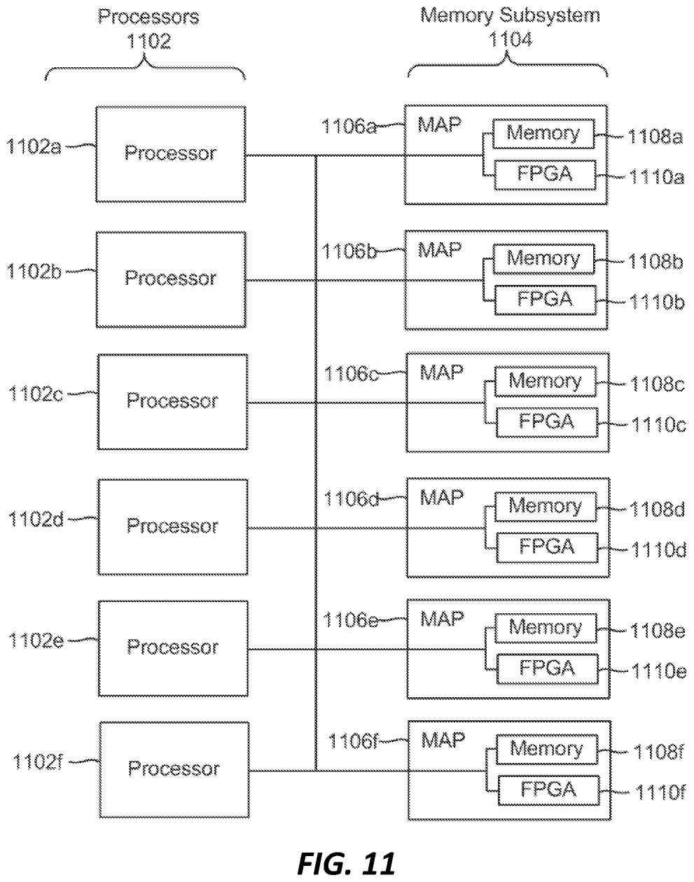

FIG. 11 is a block diagram of a multiprocessor computer system 1100 using a shared virtual address memory space in accordance with an example embodiment. The system includes a plurality of processors 1102a-f that can access a shared memory subsystem 1104. The system incorporates a plurality of programmable hardware memory algorithm processors (MAPs) 1106a-f in the memory subsystem 1104. Each MAP 1106a-f can comprise a memory 1108a-f and one or more field programmable gate arrays (FPGAs) 1110a-f. The MAP provides a configurable functional unit and particular algorithms or portions of algorithms can be provided to the FPGAs 1110a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 1108a-f, allowing it to execute tasks independently of, and asynchronously from, the respective microprocessor 1102a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card.

### EMBODIMENTS

Provided herein are methods for storing information, comprising: a) providing a structure comprising a surface; b) depositing at least one nucleotide on the surface, wherein the at least one nucleotide couples to a polynucleotide attached to the surface; and c) repeating step b) to synthesize a plurality of polynucleotides on the surface, wherein a storage density of unique polynucleotides on the surface is at least 100×10⁶ polynucleotides per cm². Further provided herein are methods, wherein the method further comprises cleaving at least one polynucleotide from the surface, wherein the polynucleotide is dissolved in a droplet. Further provided herein are methods, wherein the method further comprises sequencing at least one polynucleotide from the surface. Further provided herein are methods, wherein the method further comprises drying the surface. Further provided herein are methods, wherein the method further comprises washing the nucleotides away from the surface. Further provided herein are methods, wherein the surface is a solid support. Further provided herein are methods, wherein the surface comprises glass, fuse silica, silicon, silicon dioxide, silicon nitride, plastics, metals, or combinations thereof.

Provided herein are methods for storing information, comprising: a) providing a structure comprising a surface; b) depositing at droplet comprising at least one nucleotide on the surface, wherein the at least one nucleotide couples to a polynucleotide attached to the surface; and c) repeating step b) to synthesize a plurality of polynucleotides on the surface, wherein the droplet has a volume of less than about 100 femtoliters.

Provided herein are methods for storing information, comprising: a) providing a structure comprising a surface; b) depositing at least one nucleotide on the surface, wherein the at least one nucleotide couples to a polynucleotide attached to the surface; and c) repeating step b) to synthesize a plurality of polynucleotides on the surface, wherein the time to repeat step b) using four different nucleotides is less than about 100 milliseconds. Further provided herein are methods, wherein the method further comprises one or more wash steps. Further provided herein are methods, wherein the method further comprises deblocking, oxidizing, washing, capping, or any combination thereof.

Provided herein are devices for storing information, comprising: a chip comprising an array of addressable loci, wherein one or more addressable loci comprise at least one electrode, a synthesis surface, and at least one fluid port, wherein the synthesis surface at each addressable loci comprises at least one polynucleotide extending from the surface, wherein a density of addressable loci on the chip is at least 100×10⁶ polynucleotides per cm². Further provided herein are devices, wherein the at least one polynucleotide is about 150 to about 500 bases in length. Further provided herein are devices, wherein the at least one polynucleotide are about 200 bases in length. Further provided herein are devices, wherein the device further comprises a reagent reservoir. Further provided herein are devices, wherein the device further comprises a heating or cooling unit. Further provided herein are devices, wherein at least one addressable locus comprises a droplet. Further provided herein are devices, wherein the droplet is less than 50 micrometers in diameter.

Provided herein are methods for storing information, the method comprising: a) converting at least one item of information in a form of at least one digital sequence to at least one nucleic acid sequence; b) synthesizing a plurality of polynucleotides having predetermined sequences collectively encoding for the at least one nucleic acid sequence; c) depositing at droplet comprising at least one nucleotide on a surface, wherein the at least one nucleotide couples to a polynucleotide attached to the surface; d) repeating step c) to synthesize the plurality of polynucleotides on the surface; and e) storing the plurality of polynucleotides, wherein the droplet has a volume of less than about 100 femtoliters.

Provided herein are methods for storing information, the method comprising: a) converting at least one item of information in a form of at least one digital sequence to at least one nucleic acid sequence; b) synthesizing a plurality of polynucleotides having predetermined sequences collectively encoding for the at least one nucleic acid sequence; c) depositing at droplet comprising at least one nucleotide on a surface, wherein the at least one nucleotide couples to a polynucleotide attached to the surface; d) repeating step c) to synthesize the plurality of polynucleotides on the surface; and e) storing the plurality of polynucleotides, wherein the time to repeat step c) using four different nucleotides is less than about 100 milliseconds.

Provided herein are methods of synthesizing polynucleotides, comprising: a) providing a structure comprising a surface, wherein the surface comprises a plurality of loci for nucleotide extension; and b) synthesizing a plurality of polynucleotides extending from the surface, wherein synthesizing comprises depositing one or more reagents by applying a potential to the surface. Further provided are methods, wherein the potential is an electric potential. Further provided are methods, wherein the surface is a solid support.

Provided herein are devices for information storage using any one of the methods described herein. Provided herein are systems for information storage using any one of the methods described herein.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A cleaning process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Highly Accurate DNA-Based Information Storage and Assembly

Digital information was selected in the form of binary data totaling about 0.2 GB included content for the Universal Declaration of Human Rights in more than 100 languages, the top 100 books of Project Guttenberg and a seed database. The digital information was encrypted into a nucleic acid-based sequence and divided into strings. Over 10 million non-identical polynucleotides, each corresponding to a string, were synthesized on a rigid silicon surface. Each non-identical polynucleotide was under equal or less than 200 bases in length. The synthesized polynucleotides were collected and sequenced and decoded back to digital code, with 100% accuracy for the source digital information, compared to the initial at least one digital sequence.

### Example 3: High Density Information Storage System

Polynucleotides are de novo synthesized by methods described herein. Following synthesis, the polynucleotides are collected into a single droplet and transferred to storage on a silicon solid support. The solid support has dimensions of 86 mm×54 mm and is 1-2 mm thick. The capacity of the solid support is 1-10 petabytes (PB) that is implemented as an addressable array of 10 gigabyte packets. The physical partitioning into packets is redundantly encoded as a leading sequence with each polynucleotide within a packet sharing a common initial sequence and any other sequence information for indexing or searching. Packets are implemented as aqueous droplets with dissolved polynucleotides, with physical redundancy of 100-1000 copies of each polynucleotide. The polynucleotide length is in a range of 100-1000 bases. Droplet volume is equivalent to spheres 40-50 μm in diameter. The solid support further comprises up to 10,000×10,000 positions in an area less than a square inch.

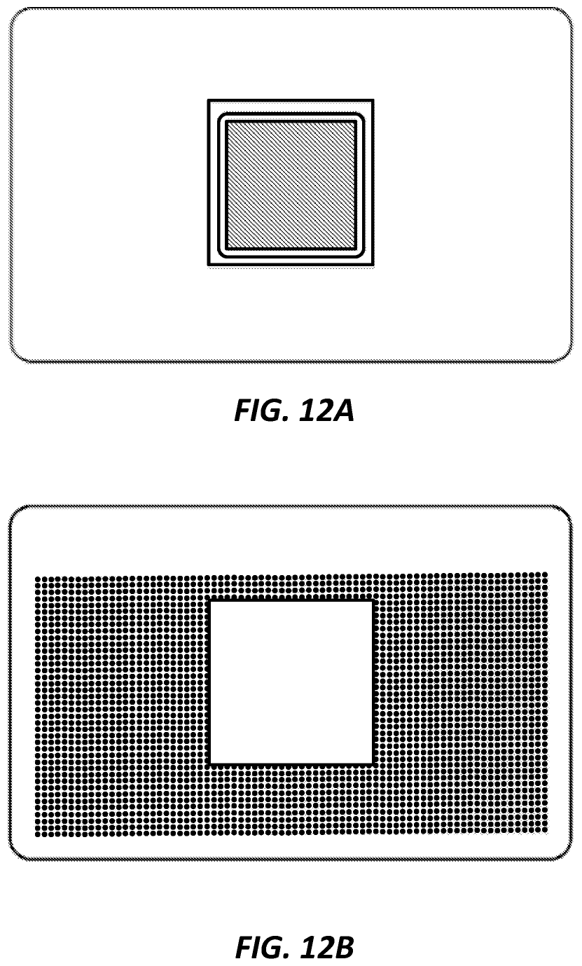

An exemplary solid support can be seen in FIGS. 12A-12B. FIG. 12A shows a front side of the solid support made of glass and comprising a clear window for array and fluidic ports. FIG. 12B shows a back side of the solid support that is a circuit comprising electrical contacts (LGA 1 mm pitch) and a thermal interface under the solid support area.

Following addition of the droplets to the solid support, the solid support may be dried and later resolvated for use for downstream applications. Alternatively, the solid support is dried and stored. Because the droplets within each packet comprise sequence information for indexing and searching, specific packets are retrieved from the plurality of packets based on the sequence information.

### Example 4: High Density Information Storage System with Access

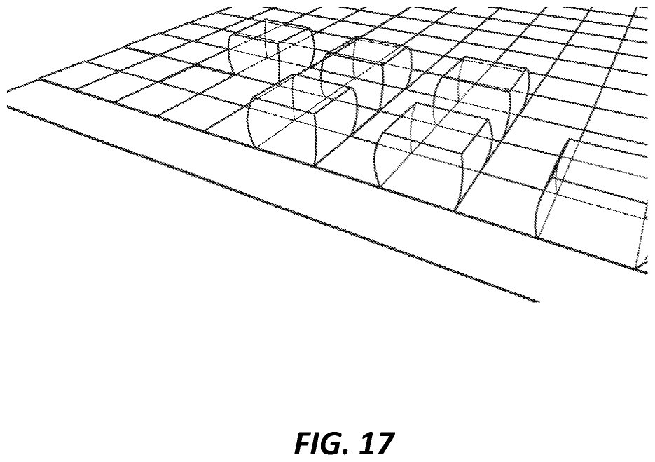

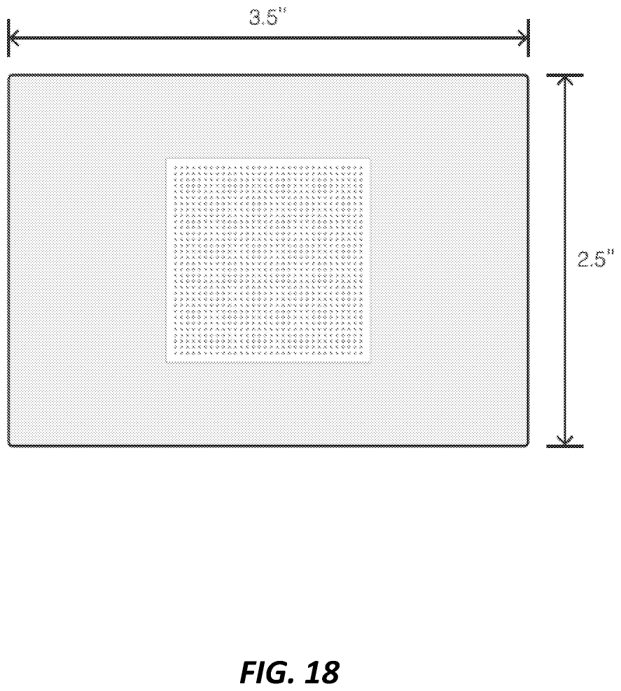

Polynucleotides are de novo synthesized by methods described herein. Following synthesis, the polynucleotides are collected into a single droplet and transferred to storage on a paper solid support. The solid support has dimensions of 3.5 inches by 2.5 inches. The capacity of the solid support is 1 petabyte that is implemented as an addressable 32×32 array comprising 1024 spots. Each spot comprises 1 terabyte pool. See e.g. FIG. 17 and FIG. 18.

At least one item of information of 10-100 megabytes is encoded in DNA and stored in 1 petabyte of data. At a later time, the 10-100 megabytes of encoded DNA is retrieved by random access of the encoded DNA and retrieving the encoded DNA from 1 terabyte pool.

### Example 5: Local Control of Polynucleotide Synthesis on a Solid Support

Polynucleotides of 240 bases in length are synthesized on a solid support using the methods described herein. Polynucleotides comprising dsDNA are approximately 80 nm in length, and polynucleotides comprising ssDNA are approximately 160 nm in length. The solid support comprises an array of 500 nm (depth)×400 nm (diameter) wells (volume approximately 0.628 femtoliters). Each wells comprises an addressable locus, addressable electrodes inside the sidewalls of each well (area is 50,000 nm²/electrode), and a 250 nm (diameter) surface for polynucleotide synthesis/attachment in addressable communication with a 250 nm (diameter) addressable bottom electrode. Electrodes are independently addressable.

Polynucleotides are present on the synthesis surface at a density of 1 polynucleotide per 50 nm². Wells are separated by a pitch of 1.0 um. 10 nm thick sidewall electrodes (located about 100 nm above the polynucleotide surface) are charged to generate a gradient of H⁺ ions that remove protecting groups (wherein the polynucleotide is blocked with an acid-cleavable blocking group) from the 5′ OH groups on defined polynucleotides at loci on the synthesis surface. H⁺ ions are then removed. (See FIG. 16B). A nucleoside phosphoramidite monomer is added, and the polynucleotides at unblocked sites and will be available for coupling to nucleosides. Cycles of deprotection and coupling are repeated to synthesis the polynucleotides. By applying a series of electrode-controlled masks to the surface before addition of each type of monomer, the desired polynucleotides are synthesized at exact locations on the surface in a controlled sequence.

### Example 6: Local Control of Polynucleotide Synthesis on a Solid Support with Electric Fields

Polynucleotides of 240 bases in length are synthesized on a solid support using the methods described herein. The solid support comprises an array of 500 nm (depth)×500 nm (diameter) wells. Each wells comprises an addressable locus, addressable electrodes inside the sidewalls of each well (area is 50,000 nm²/electrode), and a 250 nm (diameter) surface for polynucleotide synthesis/attachment in addressable communication with a 250 nm (diameter) addressable bottom electrode. Electrodes are independently addressable.

Polynucleotides are present on the synthesis surface at a density of 1 polynucleotide per 50 nm². Wells are separated by a pitch of 1.0 um. A nucleoside phosphoramidite monomer is added, and the polynucleotides at unblocked sites and will be available for coupling to nucleosides. Cycles of deprotection and coupling are repeated to synthesis the polynucleotides. By applying a series of electrode-controlled masks to the surface before addition of each type of monomer, the desired polynucleotides are synthesized at exact locations on the surface in a controlled sequence. Bottom electrodes in the bottom of the well are activated, which induces release of the polynucleotides attached thereto. Sidewall electrodes are charged to generate an electric field which moves the polynucleotides out of the well. Nucleoside phosphoramidite monomers are then added which extend from reusable linkers attached to the surface, and synthesis is repeated.

### Example 7: Local Control of Polynucleotide Synthesis on a Solid Support

Polynucleotides of 240 bases in length are synthesized on a solid support using the methods described herein. Polynucleotides comprising dsDNA are approximately 80 nm in length, and polynucleotides comprising ssDNA are approximately 160 nm in length. The solid support comprises an array of 100 nm diameter addressable electrodes on the surface for polynucleotide synthesis/attachment (See FIG. 19).

Polynucleotides are present on the synthesis surface at a density of 1 polynucleotide per 39.27 nm². Wells are separated by a pitch of 0.25 um. The electrodes are charged to generate a gradient of H⁺ ions that remove protecting groups (wherein the polynucleotide is blocked with an acid-cleavable blocking group) from the 5′ OH groups on defined polynucleotides at loci on the synthesis surface. H⁺ ions are then removed. A nucleoside phosphoramidite monomer is added, and the polynucleotides at unblocked sites and will be available for coupling to nucleosides. Cycles of deprotection and coupling are repeated to synthesis the polynucleotides. By applying a series of electrode-controlled masks to the surface before addition of each type of monomer, the desired polynucleotides are synthesized at exact locations on the surface in a controlled sequence.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-68. (canceled)

69. A device, comprising:
a solid support comprising a plurality of wells, wherein each well comprises:
a synthesis surface located at a bottom region of the well;
a bottom electrode;
a sidewall electrode located on a sidewall of the well; and
a polynucleotide extending from the synthesis surface;
wherein the solid support includes at least 1.0×10⁸ polynucleotides extending from synthesis surfaces per cm².

70. The device of claim 69, wherein the plurality of wells comprises a first well, and the polynucleotide extending from the synthesis surface of the first well has a sequence that is different than polynucleotides of the other wells of the plurality of wells.

71. The device of claim 69, wherein each of the wells comprises a depth of 100 nm to 1000 nm.

72. The device of claim 69, wherein each of the wells comprises a longest cross-sectional diameter of 100 nm to 800 nm.

73. The device of claim 69, wherein each of the wells is cylindrical.

74. The device of claim 69, wherein the bottom electrode has a cross-sectional area of 10⁴ nm² to 10⁵ nm².

75. The device of claim 69, wherein the sidewall electrode is 50 nm to 200 nm from the synthesis surface.

76. The device of claim 69, wherein the at least one sidewall electrode comprises a height of 5 nm to 25 nm.

77. The device of claim 69, wherein the sidewall electrode is a first sidewall electrode, and wherein each well further comprises a second sidewall electrode.

78. The device of claim 69, wherein the sidewall electrode and the bottom electrode are independently addressable.

79. A device, comprising:
a solid support comprising a plurality of wells, wherein each well comprises:
a synthesis surface located at a bottom region of each of the wells;
a bottom electrode; and
at sidewall electrode located on a sidewall, wherein the sidewall electrode and the bottom electrode are independently addressable, and wherein the sidewall electrode is 50 nm to 200 nm from the bottom region.

80. The device of claim 79, wherein the solid support comprises addressable loci at a density of at least 1.00×10⁸ addressable loci per cm².

81. The device of claim 79, wherein each of the wells comprises a depth up to about 1000 nm.

82. The device of claim 79, wherein each of the wells comprises a longest cross-sectional diameter of 100 nm to 800 nm.

83. The device of claim 79, wherein each of the wells is cylindrical.

84. The device of claim 79, wherein the bottom electrode comprises a longest cross-sectional area of 10⁴ nm² to 10⁵ nm².

85. The device of claim 79, wherein the sidewall electrode is 75 nm to 125 nm from the bottom region.

86. The device of claim 79, wherein the sidewall electrode comprises a height of 5 nm to 25 nm.

87. The device of claim 79, comprising at least two sidewall electrodes.

88. The device of claim 87, wherein the at least two sidewall electrodes are independently addressable.
